(19) United States
(12) Patent Application Publication
(10) Pub. No.: US 2025/0235422 A1
(43) Pub. Date: Jul. 24, 2025

(54) DODECA-2,4,6,8,10-PENTAENOIC ACID COMPOSITIONS AND USES THEREOF

(71) Applicant: BiomEdit, Inc., Fishers, IN (US)

(72) Inventors: Dharanesh Gangaiah, Fishers, IN (US); Arvind Kumar, Fishers, IN (US); Eric Nungester, Fishers, IN (US)

(21) Appl. No.: 19/036,311

(22) Filed: Jan. 24, 2025

Related U.S. Application Data
(60) Provisional application No. 63/624,335, filed on Jan. 24, 2024.

(51) Int. Cl. A61K 31/202; A61K 9/00; A61K 9/107; A61K 33/00; A61K 33/30; A61K 35/00; A61K 35/747; A61P 17/00; A61P 17/16; A61P 29/00

(52) U.S. Cl. CPC A61K 31/202; A61K 9/0014; A61K 9/107; A61K 33/00; A61K 33/30; A61K 35/747; A61P 17/00; A61P 17/16; A61P 29/00; A61K 2035/115

(57) ABSTRACT

The present invention provides compositions of Dodeca-2,4,6,8,10-pentaenoic acid (DDPA) prepared as a composition emulsion for topical use and methods for improving animal skin health. The compositions are useful for treating an inflammatory skin condition by the topical application of an emulsion as an ointment, a cream, a foam, or a gel.

### CROSS-REFERENCE TO RELATED APPLICATIONS

The present application claims priority to U.S. Application Ser. No. 63/624,335, filed Jan. 24, 2024, the entire contents of which is incorporated by reference herein.

### FIELD OF THE INVENTION

The present invention relates to Dodeca-2,4,6,8,10-pentaenoic acid compositions and methods for improving animal health. The Dodeca-2,4,6,8,10-pentaenoic acid compositions alleviate the effects of.

### BACKGROUND OF THE INVENTION

Direct fed microbials (DFMs), often also called probiotics, are microorganisms which colonize, at least temporarily, the gastrointestinal tract of an animal and provide some beneficial effect to that animal. Beneficial activities provided by a DFM can be the synthesis of vitamins or other nutritional molecules needed for a healthy metabolism of the host animal. A DFM or probiotic can also protect the host animal from disease, disorders, or clinical symptoms caused by other, pathogenic microorganisms. For example, the DFM or probiotic may produce factors having inhibitory or cytotoxic activity against certain species of pathogens, such as deleterious or disease-causing bacteria, or immunomodulatory activity, such as modulating immune response or improving immune response to foreign agent(s) or foreign antigen. The DFM or probiotic may produce factors or biomolecules having therapeutic activity, including blocking, or reducing inflammation or inflammatory response(s).

The animal hosts can recognize microbial metabolites via different pathways, including via pattern recognition receptors and the aryl hydrocarbon receptor (AhR) (Takeuchi O, Akira S (2010) Cell 140:805-820; doi.org/10.1016/j.cell.2010.01.022; Moura-Alves P et al (2014) Nature 512:387-392; doi.org/10.1038/nature13684). The role of AhR has been extensively studied in relation to metabolism of environmental toxins, but the focus has recently shifted to its role in modulation of the adaptive and innate immune system. AhR is a ligand activated transcription factor that plays a key role in a variety of diseases, including amelioration of inflammation in the gut and skin.

AHR is a transcription factor activated by various environmental ligands of plant or microbial origin. AHR activation by appropriate ligands up-regulates expression of various gene products that either play an essential role in strengthening the epidermal barrier function (FLG, LOR, and IVL) or help neutralize disease-associated ROS response (NRF2). AHR activation exerts an anti-inflammatory effect on the immune system. The signal transduction pathway of AhR is presented in FIG. 1. See Barroso A et al., 2021, Cellular Mol Immunol 18:259-268.

An inactive form of AHR is localized in the cytosol in a complex composed of HSP90, AIP, p23, and c-SRC. AHR agonists induce conformational changes in AHR that result in its translocation to the nucleus. In the nucleus, AHR interacts with ARNT, and the heterodimer is responsible for the transcription of XRE-containing genes.

AHR ligands show a high level of structural diversity with most of the ligands exhibiting agonist activity. See Table 1 reproduced from Barroso A et al., 2021, Cellular Mol Immunol 18:259-268.

| Ligand | Activity | Source |
|---|---|---|
| 1,4-dihydroxy-2-naphthoic acid (DHNA) | Agonist | Microbial |
| C1═CC═C2C(═C1)C(═CC(═C2O)C(═O)O)O | | metabolism |
| 2-(19H-indole-39-carbonyl)-thiazole-4-carboxylic acid methyl ester | Agonist | Tryptophan |
| (ITE) | | metabolism |
| COC(═O)C1═CSC(═N1)C(═O)C2═CNC3═CC═CC═C32 | | |
| 2-(Indol-3-ylmethyl)-3,39-diindolylmethane (Ltr-1) | Agonist | Cruciferous |
| | | vegetables |
| 2,3,7,8-Tetrachlorodibenzop-dioxin (TCDD) | Agonist | Anthropogenic |
| C1═C2C(═CC(═C1Cl)Cl)OC3═CC(═C(C═C3O2)Cl)Cl | | |
| 3,3-diindolylmethane (DIM) | Agonist | Cruciferous |
| C1═CC═C2C(═C1)C(═CN2)CC3═CNC4═CC═CC═C43 | | vegetables |
| 3′4′-Dimethoxyflavone | Partial | Anthropogenic |
| (DMF)COC1═CC═C(C═C1)C2═C(C(═O)C3═CC═CC═C3O2)OC | Agonist | |
| 3-Methylcholanthrene | Agonist | Anthropogenic |
| CC1═CC═C2C═C3C4═CC═CC═C4C═CC3═C5CCC1═C52 | | |
| 3-Methylindole (Skatole) | Partial | Tryptophan |
| CC1═CNC2═CC═CC═C12 | Agonist | metabolism |
| 4-hydroxy-tamoxifen (4OHT). | Agonist | Anthropogenic |
| CC/C(═C(\C1═CC═CC═C1)/C2═CC═C(C═C2)OCCN(C)C)/C3═CC═C(C═C3)O | | |
| 5-hydroxytryptophan (5HTP) | Agonist | Natural |
| C1═CC2═C(C═C1O)C(═CN2)CC(C(═O)O)N | | amino acid |
| 6-Formylindolo [3,2-b] carbazole (FICZ) | Agonist | Tryptophan |
| C1═CC═C2C(═C1)C3═CC4═C(C5═CC═CC═C5N4)C(═C3N2)C═O | | metabolism |
| 6-Methyl-1,3,8-trichlorodibenzofuran (6-MCDF) | Partial | Anthropogenic |
| CC1═CC(═CC2═C1OC3═C2C(═CC(═C3)Cl)Cl)Cl | Agonist | |
| Baicalin | AntAgonist | Scutellaria |
| C1═CC═C(C═C1)C2═CC(═O)C3═C(C(═C(C═C3O2)O[C@H]4[C@@H] | | baicalensis |
| ([C@H]([C@@H]([C@H](O4)C═O)O)O)O)O)O)O | | (plant) |
| Beta-naphthoflavone | Agonist | Anthropogenic |
| C1═CC═C(C═C1)C2═CC(═O)C3═C(O2)C═CC4═CC═CC═C43 | | |
| Bilirubin | Agonist | Heme |
| CC1═C(NC(═C1CCC(═O)O)CC2═C(C(═C(N2)/C═C\3/ | | metabolism |
| C(═C(C(═O)N3)C)C═C)C)CCC(═O)O)/C═C\4/C(═C(C(═O)N4)C═C)C | | |
| Biliverdin | Agonist | Heme |
| CC\1═C(/C(═C/C2═C(C(═C(N2)/C═C\3/C(═C(C(═O)N3)C)C═C)C)CCC(═O)O)/ | | metabolism |
| N/C1═C\C4═NC(═O)C(═C4C)C═C)CCC(═O)O | | |
| CH-223191 | AntAgonist | Anthropogenic |
| CC1═CC═CC═C1N═NC2═CC(═C(C═C2)NC(═O)C3═CC═NN3C)C | | |
| Cinnabarinic acid (CA) | Agonist | Tryptophan |
| C1═CC(═C2C(═C1)OC3═CC(═O)C(═C(C3═N2)C(═O)O)N)C(═O)O | | metabolism |
| Curcumin | Agonist | Natural |
| COC1═C(C═CC(═C1)/C═C/C(═O)CC(═O)/C═C/C2═CC(═C(C═C2)O)OC)O | | pigment |
| Diclofenac | Agonist | Anthropogenic |
| C1═CC═C(C(═C1)CC(═O)O)NC2═C(C═CC═C2Cl)Cl | | |
| Diosmin | Agonist | Citrus fruit |
| C[C@H]1[C@@H]([C@H]([C@H]([C@H](O1)OC[C@@H]2[C@H] | | peel |
| ([C@@H]([C@H]([C@@H] | | |
| (O2)OC3═CC(═C4C(═C3)OC(═CC4═O)C5═CC(═C(C═C5)OC)O)O)O)O)O)O)O)O | | |
| Gallic acid | Agonist | Different |
| C1═C(C═C(C═C1O)O)O)C(═O)O | | plants |
| GNF351 | AntAgonist | Anthropogenic |
| CC1═CC(═CN═C1)C2═NC(═C3C(═N2)N(C═N3)C(C)C)NCCC4═CNC5═CC═CC═C54 | | |
| Hydroxyeicosatrienoic acid ([12(R)-HETE]) | Agonist | Arachidonic |
| CCCCC/C═C\C[C@H](/C═C/C═C\C/C═C\CCCC(═O)O)O | | acid |
| | | metabolism |
| Indigo | Agonist | Indigofera |
| C1═CC═C2C(═C1)C(═C(N2)C3═NC4═CC═CC═C4C3═O)O | | spp (plant) |
| Indirubin | Agonist | Indigofera |
| C1═CC═C2C(═C1)C(═C(N2)O)C3═NC4═CC═CC═C4C3═O | | spp (plant) |
| Indole | Agonist | Diverse |
| C1═CC═C2C(═C1)C═CN2 | | natural |
| | | origen |
| Indole-3-acetic acid (IAA) | Agonist | Plant |
| C1═CC═C2C(═C1)C(═CN2)CC(═O)O | | hormone |
| | | (Indole |
| | | derivative) |
| Indole-3-acetonitrile (IAN) | Agonist | Plant |
| C1═CC═C2C(═C1)C(═CN2)CC#N | | hormone |
| | | (indole |
| | | derivative) |
| Indole-3-aldehyde (I3CA) | Agonist | Tryptophan |
| C1═CC═C2C(═C1)C(═CN2)C═O | | metabolism |
| | | by bacterias |
| Indole-3-carbinol (I3C) | Agonist | Cruciferous |
| C1═CC═C2C(═C1)C(═CN2)C═O | | vegetables |
| Indolo [3,2-b]carbazole (ICZ) | Agonist | Indole-3- |
| C1═CC═C2C(═C1)C3═CC4═NC5═CC═CC═C5C4═CC3═N2 | | carbinol |
| Indoxyl-3-sulfate (I3S) | Agonist | Tryptophan |
| C1═CC═C2C(═C1)C(═CN2)OS(═O)(═O)O | | metabolism |
| Kynurenic acid (KA) | Agonist | Tryptophan |
| C1═CC═C2C(═C1)C(═O)C═C(N2)C(═O)O | | metabolism |
| L-Kynurenine (Kyn) | Agonist | Tryptophan |
| C1═CC═C(C(═C1)C(═O)C[C@@H](C(═O)O)N)N | | metabolism |
| Laquinimod | Agonist | Anthropogenic |
| CCN(C1═CC═CC═C1)C(═O)C2═C(C3═C(C═CC═C3Cl)N(C2═O)C)O | | |
| Lipoxin A4 | Agonist | Arachidonic |
| CCCCC[C@@H](/C═C/C═C\C═C\C═C\[C@H]([C@H] | | acid |
| (CCCC(═O)O)O)O)O | | metabolism |
| Malassezin | Agonist | Malassezia |
| C1═CC═C2C(═C1)C(═CN2)CC3═C(C4═CC═CC═C4N3)C═O | | furfur |
| | | (fungi) |
| 3′-methoxy-4′-nitroflavone (MNF) | Antagonist | Shynthetic |
| COC1═C(C═CC(═C1)C2═CC(═O)C3═CC═CC═C3O2)[N+](═O)[O−] | | falvone |
| | | derivative |
| Norisoboldine | Agonist | Lindera |
| COC1═C(C2═C3[C@H](CC4═CC(═C(C═C42)OC)O)NCCC3═C1)O | | aggregata |
| | | (plant) |
| Omeprazole | Agonist | Anthropogenic |
| CC1═CN═C(C(═C1OC)C)CS(═O)C2═NC3═C(N2)C═C(C═C3)OC | | |
| Prostaglandin | Agonist | Arachidonic |
| CCCCCC(/C═C/C1C═CC(═O)C1C/C═C/CCCC(═O)OC)OC(═O)C | | acid |
| | | metabolism |
| Quercetin | Partial | Fruits and |
| C1═CC(═C(C═C1C2═C(C(═O)C3═C(C═C(C═C3O2)O)O)O)O)O | agonist | vegetables |
| (complete) | | |
| StemRegenin 1 (SR1) | AntAgonist | Purine |
| CC(C)N1C═NC2═C(N═C(N═C21)C3═CSC4═CC═CC═C43)NCCC5═CC═C(C═C5)O | | derivative |
| Sulindac | Agonist | Anthropogenic |
| CC\1═C(C2═C(/C1═C\C3═CC═C(C═C3)S(═O)C)C═CC(═C2)F)CC(═O)O | | |
| Resveratrol | Partial | Fruits and |
| C1═CC(═CC═C1/C═C/C2═CC(═CC(═C2)O)O)O | agonist | vegetables |
| Tapinarof | Agonist | Bacterial |
| CC(C)C1═C(C═C(C═C1O)/C═C/C2═CC═CC═C2)O | | metabolism |
| Tryptamine | Agonist | Tryptophan |
| C1═CC═C2C(═C1)C(═CN2)CCN | | metabolism |
| Tryptanthrin | Agonist | Tryptophan |
| C1═CC═C2C(═C1)C(═O)N3C4═CC═CC═C4C(═O)C3═N2 | | metabolism |
| VAF347 | Agonist | Anthropogenic |
| C1═CC(═CC(═C1)C1)C2═NC(═NC═C2)NC3═CC═C(C═C3)C(F)(F)F | | |
| Xanthurenic acid | Agonist | Tryptophan |
| C1═CC2═C(C(═C1)O)NC(═CC2═O)C(═O)O | | metabolism |
| AHR antagonist 5 (IK-775) | Antagonist | |
| FC1═CN═CC(C2═NC3═C(C(C)C)C═NN3C(N[C@@H] | | |
| (C4)CCC5═C4C6═C(N5)C═CC═C6)═N2)═C1 | | |

Skin barrier function is regulated by AHR. Not to be limited by theory, it is believed that skin barrier function is regulated via competition between AHR axis (up-regulation of barrier) and the IL-13/IL-4-JAK-STAT6/STAT3 axis (down-regulation) of barrier. IL-13/IL-4-JAK-STAT6/STAT3 axis (targeted by several therapeutic principles) down-regulates expression of FLG, LOR, and IVL and generates ROS and disruption of barrier function. AHR axis up-regulates expression of filaggrin (FLG), loricrin (LOR), and involucrin (IVL) leading to strengthened barrier function. In addition, antioxidative AHR agonists, e.g. tapinarof (WBI-1001, GSK2894512 or bentivimod; clinical phase 2*) activate nuclear factor E2-related factor 2 (NRF2) leading to ROS neutralization.

Examples of AhR agonists include tapinarof (3,5-dihydroxy-4-isopropyl-trans-stilbene; Smith, et al., J. Invest. Dermatol., 137(1):2110-2119 (2017)); 3-methyl cholanthrene, dioxin, 3′,4′-dimethoxy-a-naphthoflavone (DiMNF), 3,3′-Diindolylmethane (DEVI), resveratrol, curcumin, indigo and indigo derivatives. Other exemplary AhR agonists are described in Hubbard, T. D. et al., Drug Metabolism and Disposition, 43:1522-1535 (2015) and in WO 2012/015914, which are incorporated by reference herein. Compounds that agonize the AhR can be identified, for example, as described in WO2016/168685.

One AHR agonist, tapinarof has been shown to be effective for treatment of psoriasis and AD in humans. Bissonnette R et al., 2021, J Am Acad Dermatol 84:1059-67. Tapinarof, via AHR activation, inhibits two pro-inflammatory pathways implicated in psoriasis (Th17 cytokines: IL-17A, IL-17F) and atopic dermatitis (Th2 cytokines: IL-4, IL-5, IL-13). AHR modulation by tapinarof also increases antioxidant activity via upregulation of Nrf2 and promotes skin barrier restoration through upregulation of a number of epidermal barrier genes, including filaggrin, hornerin, and involucrin. Tapinarof is a secondary metabolite from Photorhabdus luminescens, a bioluminescent, Gram-negative bacillus, which lives symbiotically within parasitic, soil-living entomopathogenic nematodes of the genus Heterorhabditis. Tapinarof is an investigational drug; two phase 3 trials, PSOARING 1 (NCT03956355) and PSOARING 2 (NCT03983980) completed in 2020 and have been preliminarily reported (Lebwohl M, Stein Gold L, Strober B, et al. SKIN J CutaneMed. 2020;4(6):s75. doi.org/10.25251/skin.4.supp.75). NDA submitted and accepted by FDA (Aug. 10th 2021). Tapinarof is under investigational study for plaque psoriasis in adults (NCT04053387).

Here we report for the first time, dodeca-2,4,6,8,10-pentaenoic acid (DDPA) as an effective AhR agonist. DDPA has been identified a natural product found in Aspergillus fumigatus (Pubchem ID: 53834148) but no biological activities were identified. DDPA has been suggested for use as a component of formulations for intravenous injection but is not identified as a therapeutically active compound. Rather, esterified DDPA is reported as useful as an ester of a C6 to C12 fatty acid to increase the solubility of sparingly soluble active compounds for intravenous injection. See U.S. Pat. No. 4,711,902. Biological activities are not previously known.

There is a need for therapeutic compositions and methods that provide beneficial molecules to the skin of an animal and improve animal health, particularly in animals exhibiting inflammatory diseases of the skin including dermatitis, dermatitis, alopecia, and psoriasis, pyoderma gangrenosum, palmoplantar pustulosis, prurigo nodularis, or hidradenitis suppurativa. There is a need in the art for approaches and therapies to mediate and alleviate the effects of inflammatory skin conditions, atopic dermatitis (AD), psoriasis (Ps), seborrheic dermatitis, contact dermatitis, hidradenitis suppurativa (HS), pyoderma gangrenosum (PG), palmoplantar pustulosis (PPP), prurigo nodularis (PN), Sweet's syndrome, mutations in proline-serine-threonine phosphatase interacting protein 1 (PSTPIP1) (PAPA syndrome, PAPSH syndrome and PASH syndrome), Bechet's disease, bullous pemphigold, mucous membrane pemphigold, pemphis vulgaris, cutaneous Crohn's disease, Sjögren syndrome, systemic lupus erythematosus, pityriasis lichenoides chronica, and erythoderma.

### SUMMARY OF THE INVENTION

The present application includes, and provides for, compositions comprising Dodeca-2,4,6,8,10-pentaenoic acid (DDPA) and an at least one excipient selected from the group consisting of diluents, fillers, disintegrants, binders, lubricants, surfactants, hydrophobic vehicles, water soluble vehicles, co-solvents, emulsifiers, buffers, humectants, moisturizers, solubilizers, preservatives, colorants, plasticizers, carriers, penetration enhancers, or combinations thereof.

The application further provides, and includes, methods of treating an inflammatory skin condition in animal in need thereof comprising applying a composition comprising an emulsion of Dodeca-2,4,6,8,10-pentaenoic acid (DDPA) and one or more excipients selected from the group consisting of diluents, fillers, disintegrants, binders, lubricants, surfactants, hydrophobic vehicles, water soluble vehicles, emulsifiers, buffers, humectants, moisturizers, solubilizers, preservatives, colorants, plasticizers, carriers, excipients, or combinations thereof.

The present application further provides for methods for treating an inflammatory skin disease comprising applying a topical composition to the skin of an animal subject in need of treatment, wherein said topical composition comprises an effective amount of an emulsion of Dodeca-2,4,6,8,10-pentaenoic acid (DDPA) and a pharmaceutically acceptable carrier.

The present application further provides for methods for modifying the expression of one or more proinflammatory cytokines or one or more anti-inflammatory cytokine in the skin of an animal in need thereof comprising administering to the animal a composition comprising effective amount of an emulsion of Dodeca-2,4,6,8,10-pentaenoic acid (DDPA) and a pharmaceutically acceptable carrier.

The present application provides for compositions for topical use in treating an inflammatory skin condition comprising an emulsion of Dodeca-2,4,6,8,10-pentaenoic acid (DDPA) and one or more excipients selected from the group consisting of diluents, fillers, disintegrants, binders, lubricants, surfactants, hydrophobic vehicles, water soluble vehicles, emulsifiers, buffers, humectants, moisturizers, solubilizers, preservatives, colorants, plasticizers, carriers, excipients, or combinations thereof.

### BRIEF DESCRIPTION OF DRAWINGS

FIG. 1 depicts the AHR signaling pathway. The inactive form of AHR is localized in the cytosol in a complex composed of HSP90, AIP, p23, and c-SRC. AHR agonists induce conformational changes in AHR that result in its translocation to the nucleus. In the nucleus, AHR interacts with ARNT, and the heterodimer is responsible for the transcription of XRE-containing genes. Notes: (AHR) aryl hydrocarbon receptor, (N) N terminal motif, (C) C terminal motif, (NLS) nuclear localization signal, (bHLH) basic-helix loop helix, (PAS) Per-Arnt-Sim, (Q-rich) glutamine rich, (HSP90) heat shock protein 90, (AIP) AHR-interacting protein, (XRE) xenobiotic responsive elements, (AHRR) AHR repressor, (CYP) cytochrome P450, (IDO) indoleamine 2,3-dioxygenase

FIG. 2 provides a diagram of AHR ligand dependent activation. Multiple mechanisms by which a specific ligand-activated AHR can stimulate gene expression are known. In the classical mechanism of AHR action, ligand binding stimulates AHR nuclear translocation, dimerization with the ARNT protein and the binding of the ligand:AHR:ARNT complex to its DNA binding site (the DRE) stimulates gene expression. However, the dimerization of liganded AHR with other proteins (e.g., KLF6 or RelB) results in the formation of unique protein complexes that bind to distinctly different DNA recognition sites (e.g., a ncXRE or RelBAhRE, respectively) to regulate subsets of genes not regulated by the AHR:ARNT complex. Whether ligand bound AHRs can interact with additional DNA binding partners remains to be determined but is a possibility. In addition to multiple heterodimers, the AHR has also been observed to bind to other nuclear protein complexes (e.g., estrogen receptor (ER) dimers) and function as a coactivator, enhancing gene expression by these transcription factors,

FIG. 3A and FIG. 3B present a model of ligand dependent activation of AHR responses. Reproduced from Denison MS and Faber SC, Current Opinion in Toxicology 2017, 2:124-131. Binding and activation of the AHR by structurally diverse AHR ligands produce significant differences in the overall structure of the AHR and/or its dimerization partner that result in recruitment of distinctly different coactivators to the DNA bound AHR complex and differential gene expression. Although this figure only depicts an alteration in the structure of the AHR, ligand-selective structural changes could also occur in the ARNT protein, or in any other protein(s) to which the AHR is bound (such as KLF6 or RelB) facilitating differential coactivator recruitment by a greater diversity of ligand-activated AHR complexes and even a greater diversity in gene expression responses.

FIG. 4 provides in vitro data from HepG2-Lucia cells demonstrating dose-dependent AHR activation by fractions from L. reuteri strain 3632 (LR3632) extract enriched for pks metabolite and depicts AhR activator activity of control FICZ versus L. reuteri 3632 pks metabolite (2.5 mg/ml).

FIG. 5 shows AHR activation studies. AHR activator activity was assessed in L. reuteri 3632 supernatant, cell pellet resuspended, and medium. Significant AhR activator activity was identified in the L. reuteri 3632 pellet when resuspended. The medium and supernatant had limited to no significant activity

FIG. 6 provides AhR activation studies and assessment of AhR activator activity. The cell pellet was resuspended in the same volume as the culture supernatant and a direct comparison of ligand presence in the supernatant versus the pellet resuspended was conducted. Again, the activity is demonstrated in the pellet resuspended with little activity in the L. reuteri strain 3632 0.2 μM filtered supernatant.

FIG. 7 presents the exemplary results of AhR activity testing on HepG2-Lucia™ AhR reporter cells. The AhR agonist 6-Formylindolo[3,2-b]carbazole (FICZ, 1 mg/ml final concentration) is a positive control. The test articles included L. reuteri 3632 and 3630 whole cells and cell-free supernatants and purified PKS metabolite [50 mg/ml in phosphate buffered saline (PBS)]. Following 36 h of incubation, 20 ml of the sample was mixed with 50 ml of Quanti-Luc™ and luciferase activity was measured immediately. The RLUs for whole cells, cell-free supernatants and purified PKS metabolite were normalized to either MRS media or PBS. The data represents mean±standard deviation of 3 independent experiments. ***P<0.001.

FIG. 8 presents a diagram of the skin lesion sore (SLS) assessment locations in dogs.

FIG. 9 presents a graphical representation of a representative SLS assessment on dog skin treated with L. reuteri strain 3632 (Treatment A), heat inactivated L. reuteri strain 3632 (Treatment B), placebo negative control, and Cortavance.

FIG. 10 presents the identification and characterization of the BGC encoding the DPPA PKS metabolite from L. reuteri 3632. A. L. reuteri 3630 (creamish white) and 3632 (orange pigmentation) cell pellets. B. L. reuteri 3632 plasmid map showing the location of the PKS locus (shown in blue). C. L. reuteri 3632 PKS locus showing the organization of 15 genes from pksA to pksO. D. Putative structure of the DPPA PKS metabolite as identified by NMR and HRMS. E. Proposed biosynthetic pathway to produce the DPPA PKS metabolite in L. reuteri 3632.

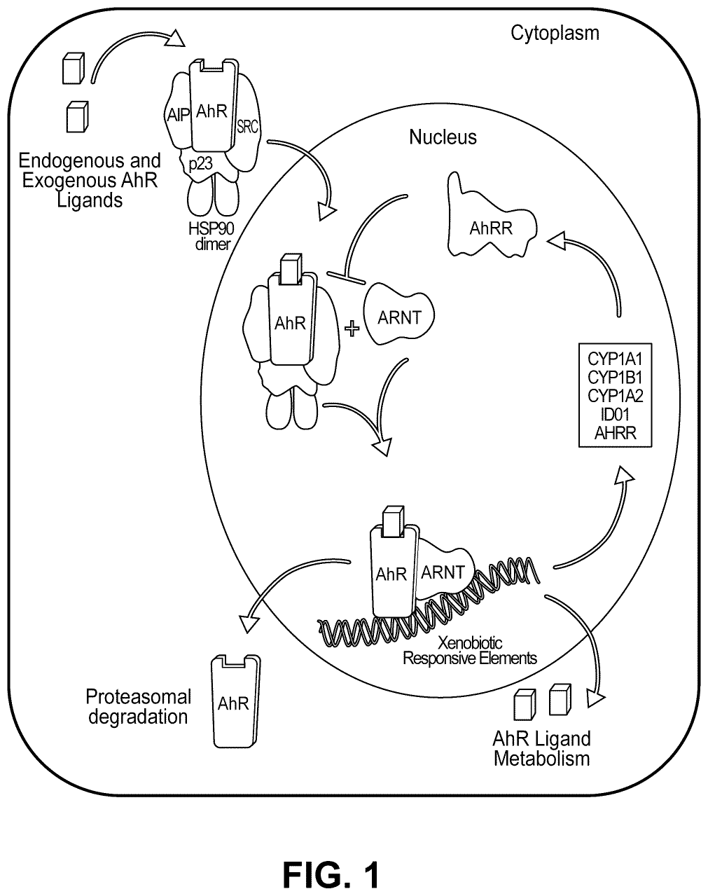

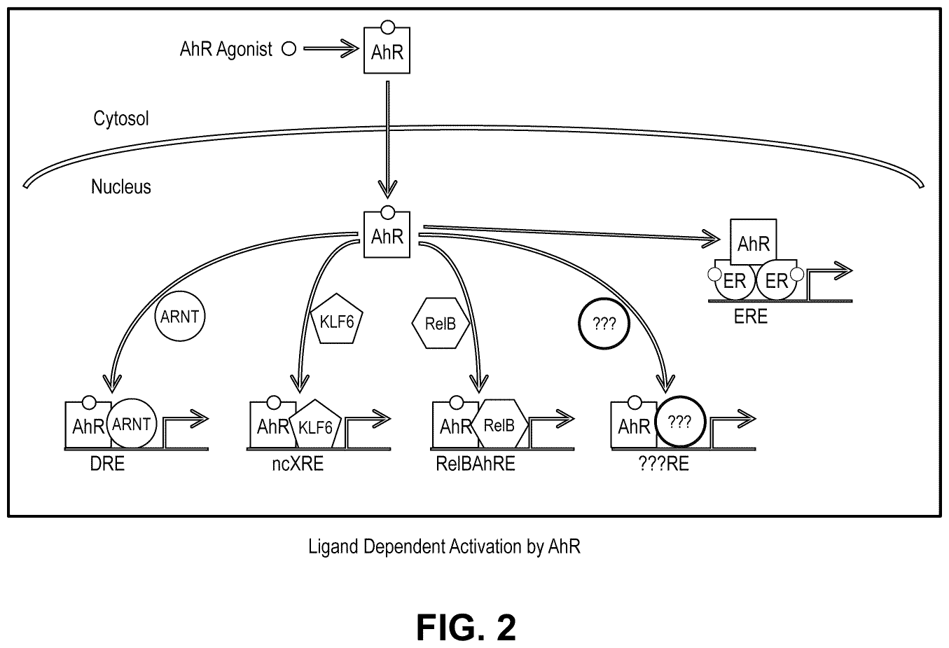

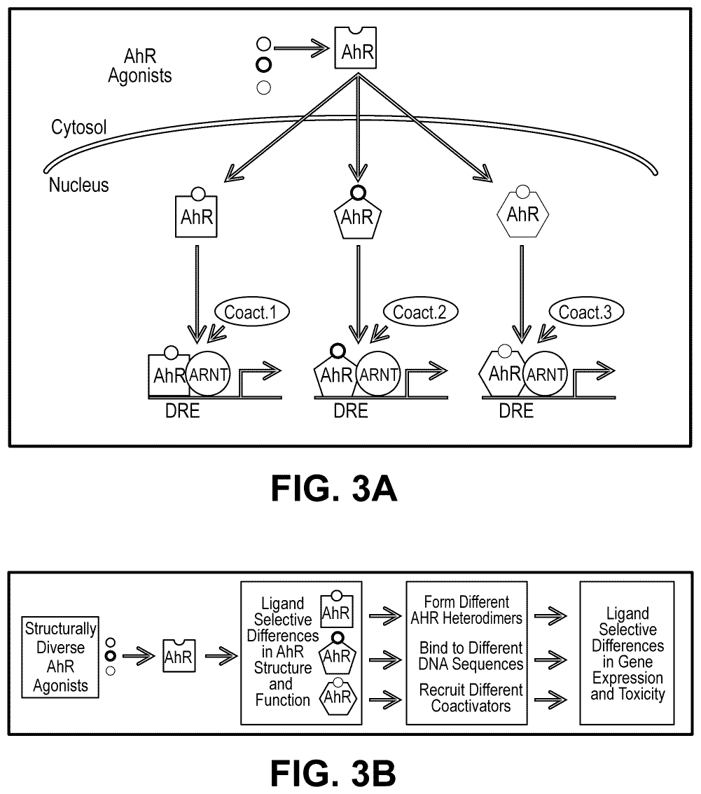

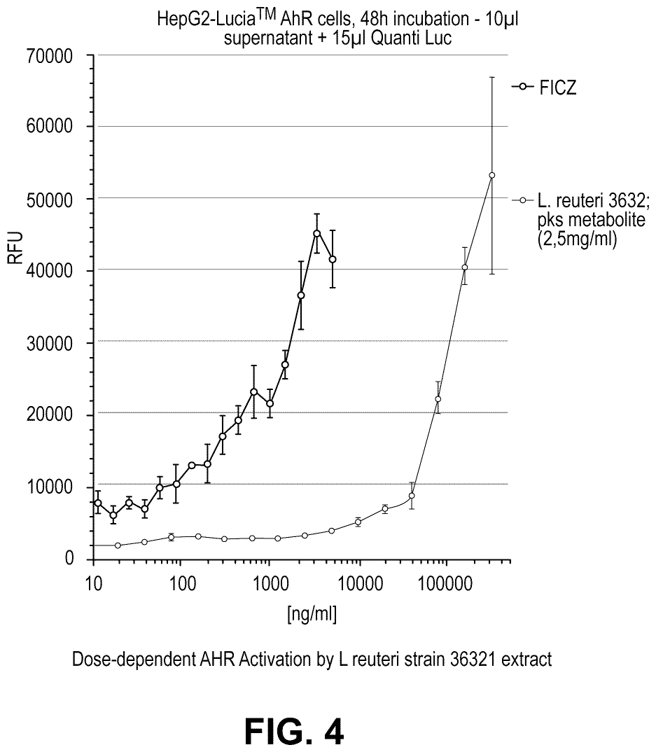

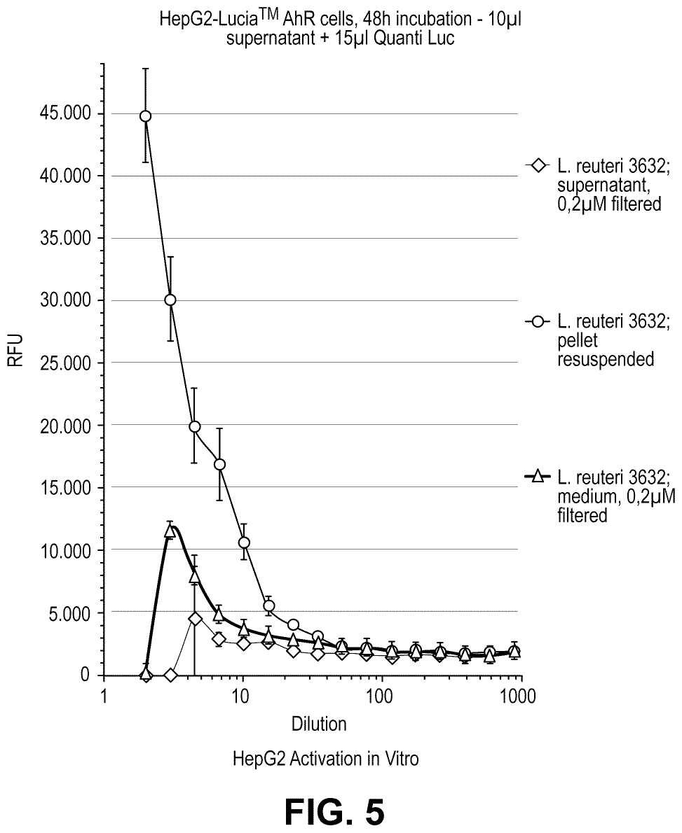

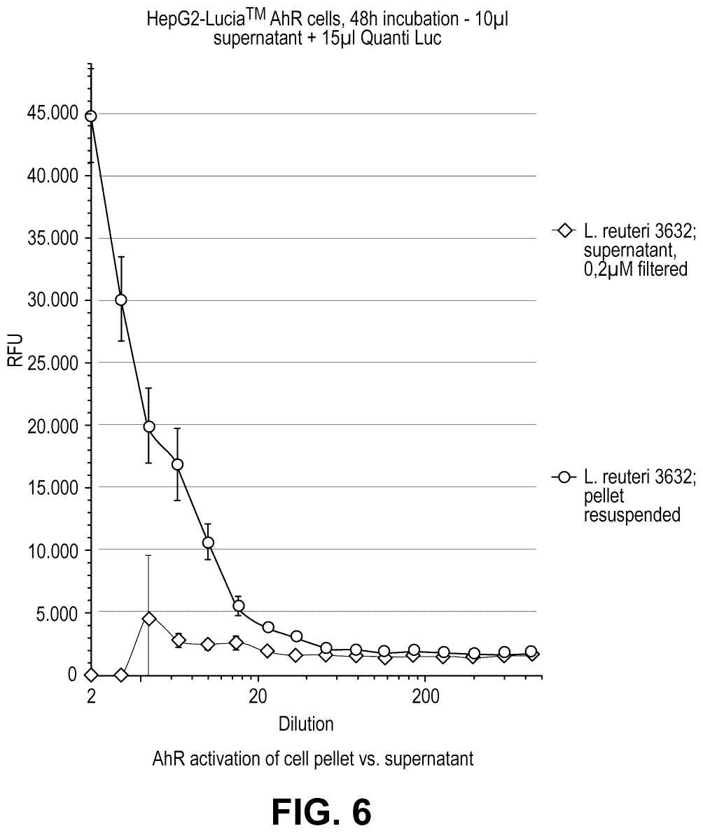

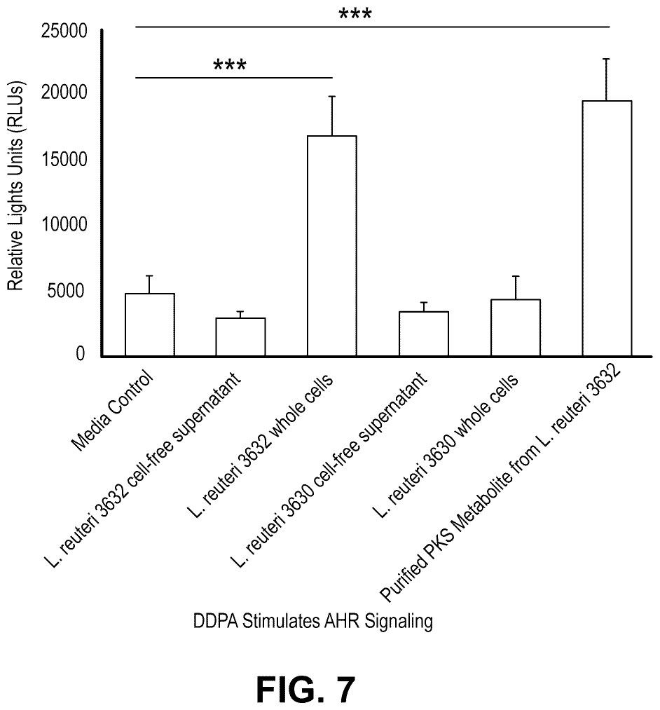

### DETAILED DESCRIPTION OF THE INVENTION

### Compositions

The present application provides for the isolation and characterization of an orange pigmented metabolite isolated from L. reuteri 3632 proposed as a product of the PKS locus. Identified as dodeca-2,4,6,8,10-pentaenoic acid (DDPA), the orange pigmented metabolite is an agonist of the AhR signaling pathway. During development, DDPA was determined to be photolabile thus requiring the isolation and storage of DDPA under low light conditions. DDPA is also hydrophobic thus complicating delivery to the skin. Topical preparations are formulated to photo-protect and stabilized for the delivery of DDPA therapeutic amounts to the skin.

The present specification provides for, and includes, compositions comprising dodeca-2,4,6,8,10-pentaenoic acid (DDPA), or salts thereof, and an acceptable excipient. The SMILES structure of DDPA is CC═CC═CC═CC═CC═CC(═O)O and the chemical structure is shown in Formula I:

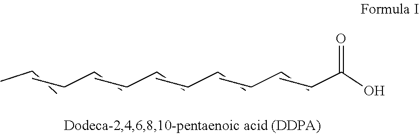

DDPA has calculated molecular weight of 190.24 grams per mole (g/mol) and is a hydrophobic compound with an XlogP3 value of 3.3. The present application shows for the first time that DDPA is an Aryl hydrocarbon Receptor (AhR) agonist.

The present specification further provides for, and includes, compositions comprising an emulsion of dodeca-2,4,6,8,10-pentaenoic acid (DDPA), or salts thereof, and one or more excipients. As an emulsion, DDPA is stabilized and deliverable at higher concentrations than the compound alone. In aspects, the emulsions are opaque emulsions thereby protecting the DDPA from photoinactivation.

As used herein, “excipient” refers to a pharmaceutically acceptable, cosmetically acceptable, or both pharmaceutically acceptable and cosmetically acceptable diluent, adjuvant, or vehicle with which the DDPA, or salt thereof, is administered.

Suitable excipients are generally known in the art and includes, but are not limited to, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and. Drug Administration as being acceptable for use in humans or domestic animals. The person of ordinary skill in the art can refer to various pharmacologic references for guidance in determining the amount of such components in the topical compositions and formulations of aspects. Suitable pharmacologic references include: Remington: The Science and Practice of Pharmacy, 20th edition, Lippincott Williams and Wilkins (2000) (ISBN: 0683306472); Introduction to Pharmaceutical Dosage Forms, 4th ed., Lea & Febiger, Philadelphia (1985); Modem Pharmaceutics, Banker & Rhodes, Marcel Dekker, Inc. (1979); Goodman & Gilman's The Pharmaceutical Basis of Therapeutics, 6th Edition, MacMillan Publishing Co, New York (1980) (ISBN: 0023447206); Jens T. Carstensen, Drug Stability: Principles & Practice, 2d. Ed., Marcel Dekker, NY, N.Y., (1995); Development and Formulation of Veterinary Dosage Forms. 2nd Ed. CRC Press. Hardee and Baggo (1998), and TFA Cosmetic Ingredient Handbook, Eleventh Edition (2006) (the “pharmacologic references”).

In aspects, emulsions of DDPA are formulated for topical application for the treatment of inflammatory skin disorders. In aspects, an emulsion of DDPA comprises an oil phase, a water phase, and a surfactant. In some aspects, the composition of DDPA is self-emulsifying. In other aspects, the emulsion is prepared by mixing to homogeneity the components. The preparation of emulsions is generally known in the art.

Topical forms provided herein include, but are not limited to, sprays, aerosols, solutions, emulsions, suspensions, eye drops or other ophthalmic preparations, or other forms known to one of skill in the art. Suitable excipients (e.g., carriers and diluents) and other materials that can be used to provide topical dosage forms encompassed herein are well known to those skilled in the pharmaceutical arts and depend on the particular tissue to which a given pharmaceutical composition or dosage form will be applied. In one aspect, excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions, or gels, which are non-toxic and pharmaceutically acceptable. Moisturizers or humectants can also be added to pharmaceutical compositions and dosage forms. Examples of additional ingredients are well known in the art. See, e.g., See, pharmacologic references, supra.

In aspects the suitable excipients are generally include one or more excipients selected from the group consisting of diluents, fillers, binders, lubricants, surfactants, hydrophobic vehicles, water soluble vehicles, emulsifiers, buffers, humectants, moisturizers, solubilizers, preservatives, colorants, plasticizers, carriers, excipients, or combinations thereof.

In aspects, the DDPA composition is prepared as an emulsion. The term “emulsion” describes a dosage form consisting of a two-phase system comprised of at least two immiscible liquids, one of which is dispersed as droplets (internal or dispersed phase) within the other liquid (external or continuous phase) generally stabilized with one or more emulsifying agents (US FDA Drug Nomenclature Monograph, number C-DRG-00201). Emulsions are formed and stabilized with the help of emulsifiers which are surfactants and/or viscosity-generating agents. A stable emulsion must contain at least three components: the first one is internal (disperse or discontinuous phase), the second one is an external (continuous) phase, and the third component is the emulsifying agent which makes a bridge between internal and external phases. In aspects, the emulsion is an oil-in-water emulsion (e.g., the oil is the disperse or discontinuous phase and water is the continuous phase) or a water-in-oil emulsion (e.g., the water is the disperse or discontinuous phase and oil is the continuous phase). In each case, the hydrophobic DDPA is solubilized in the oil phase and protected by the opaque nature of the emulsion.

In aspects, compositions comprising DDPA are formulated for topical application to the skin of an animal. In aspects, the topical compositions may be formulated in any formulation suitable for topical administration, including, but not limited to, a solution, fluid, emulsion, suspension, solid, semi-solid, jelly, paste, gel, hydrogel, ointment, lotion, emulsion, cream, foam, mousse, liquid, spray, suspension, dispersion, powder, aerosol, or transdermal patch. In certain aspects, the DDPA is formulated as an emulsion comprising an ointment, a cream, a foam, or a gel. Topical formulations of DDPA are suitable for application to the skin of an animal, as a spray, an ointment, a cream, a foam, or a gel, and useful for the preparations of medications for the treatment of inflammatory skin conditions. In aspects, the topical formulation is an ointment, cream, foam, or gel is that hydrophobic or lipophilic.

As provided herein, the topical formulations comprising DDPA provides DDPA in a therapeutically effective amount. In aspects, topical formulations comprising DDPA in an amount of 0.01% to 5% by weight of the composition are provided.

### Emulsions

In aspects, the present specification provides for a topical pharmaceutical emulsion composition comprising the active ingredient Dodeca-2,4,6,8,10-pentaenoic acid (DDPA) or a pharmaceutically acceptable salt thereof, and one or more exciptents. In aspects, the emulsion comprises an oil phase, a water phase, a surfactant, wherein the average droplet size of the discontinuous phase is less than about 35 microns. In aspects, the emulsion comprises an oil phase, a water phase, a surfactant, and a photoprotectant wherein the average droplet size of the discontinuous phase is less than about 35 microns. In aspects, the emulsion comprises an oil phase, a water phase, a surfactant, a photoprotectant, and a buffer wherein the average droplet size of the discontinuous phase is less than about 35 microns. In aspects, the emulsion comprises an oil phase, a water phase, a surfactant, a photoprotectant, and a buffer wherein the average droplet size of the discontinuous phase is less than about 35 microns. In aspects, the emulsion comprises an oil phase, a water phase, a surfactant, a photoprotectant, a chelating agent, and a buffer wherein the average droplet size of the discontinuous phase is less than about 35 microns.

### Photoprotective Compositions

While emulsions typically have an opaque and thereby photoprotective character, in some aspects, the compositions include a photoprotective substance. In aspects, the photoprotective susbstance can be inorganic oxides like zinc oxide, or an organic UV absorbers like cinnamic acid derivatives or dibenzoylmethane derivatives.

Importantly, DDPA is a photosensitive compound and photodecomposition leads to a loss of potency. Thus, when preparing DDPA (either from a biological source or through synthetic means), it needs to be protected from light. Similarly, formulations and compositions of DDPA are protected from light until use. In some aspects, a light protecting component is included as an excipient. In further aspects, as photodegradation often involves a metal acting as a catalyst, the light protecting component can be a chelating agent.

In other aspects, the DDPA is provided in a light protected form to extend the treatment period. In its simplest form, DDPA compositions can be applied and then covered with an opaque material, such as a bandage or as applied in a patch. In other aspects, the DDPA is protected by either a light absorbing, or light reflecting carrier component and allowed to penetrate the skin of a treated animal over a longer period.

In alternative aspects, the DDPA is provided in a protected container, and then applied to the skin of an animal without a light protective component. Thus, the unprotected DDPA has a reduced half-life and residence compared to light-protected compositions. As provided herein, the half-life and residence of DDPA (e.g., the effective therapeutic amount) in a clinical application can be controlled. For short acting applications, the DDPA can be applied in a spray with suitable carriers to assist in the rapid penetration of the skin. In aspects, after application, excess DDPA can be reduced by the application of light to induce photolysis.

Suitable light protective components are known in the art and include, without limitation, zinc oxide or titanium oxide. In aspects, the light protective component comprises an antioxidant, for example ascorbic acid or alpha-tocopheraol. In further aspects, the light protective components include, without limitation, monomeric polyacids such as EDTA, cyclohexanediamine tetraacetic acid (CDTA), hydroxyethylethylenediamine triacetic acid (HEDTA), diethylenetriamine pentaacetic acid (DTPA), dimercaptopropane sulfonic acid (DMPS), dimercaptosuccinic acid (DMSA), aminotrimethylene phosphonic acid (ATP A), citric acid, pharmaceutically acceptable salts thereof, and combinations of any of the foregoing. Other exemplary chelating agents include phosphates, e.g., pyrophosphates, tripolyphosphates, and hexametaphosphates; diethylene tri amine pentaacetic acid (DTPA), ethylene diamine tetraacetic acid (EDTA), nitrilotriacetic acid (NT A), 1,3-propylene diamine tetraacetic acid (PDTA), Ethylene diamine disuccinic acid (EDDS), and ethylene glycol tetraacetic acid (EGTA). The selection of an appropriate chelating agent as a photoprotective agent can be determined through routine experimentation.

Inorganic screening agents are selected from among pigments (mean size of the primary particles: generally from 5 nm to 100 nm and preferably from 10 nm to 50 nm) of coated or uncoated metal oxides, for instance nanopigments of titanium oxide (amorphous or crystallized in rutile and/or anatase form), of iron oxide, of zinc oxide, of zirconium oxide or of cerium oxide, which are all photoprotective agents that are well known.

### Chelating Agents

As discussed above, chelating agents are useful as, but not limited to, light protective agents in compositions and formulations. Chelating agents are also known to be useful to reduce microbial growth. In aspects, the DDPA compositions further include a chelating agent or as a mixture of chelating agents. Similarly, the DDPA compositions provided herein can include both a chelating agent and an antioxidant. Suitable chelating agents of the present DDPA compositions include, but are not limited to, citric acid, glucuronic acid, sodium hexametaphosphate, zinc hexametaphosphate, ethylene diamine tetraacetic acid (EDTA), phosphonates, salts thereof, and mixtures thereof. Ethylene diamine tetraacetic acid is also known as edetic acid.

In an aspect, the chelating agent is EDTA or a salt thereof, such as potassium, sodium, or calcium salts of EDTA. In an aspect, the EDTA or a salt thereof is disodium EDTA. In another aspect, the chelating agent is citric acid. In yet another aspect, the DDPA compositions comprise a mixture of a chelating agent and an antioxidant which is a mixture of EDTA or a salt thereof and propyl gallate. In a further aspect, the DDPA compositions provided herein comprise a mixture of a chelating agent and an antioxidant which is a mixture of EDTA or a salt thereof and BHT. In one aspect, the DDPA compositions and formulations provided herein comprise a mixture of a chelating agent and an antioxidant which is a mixture of disodium EDTA and BHT.

In yet a further aspect, the DDPA compositions and formulations comprise a mixture of a chelating agent and an antioxidant which is a mixture of citric acid and propyl gallate. In an aspect, the DDPA compositions and formulations comprise a mixture of a chelating agent and an antioxidant which is a mixture of citric acid and BHT.

### Buffering and Buffering Agents

As provided herein, the DDPA compositions can include as an excipient one or more buffers or buffering agents. Skin typically has a pH of between 4.7 and 5.75. Increases in pH lead to dry skin and susceptibility to eczema and infection. Overly acidic skin can lead to increased inflammation and acne.

Accordingly, in aspects, the DDPA compositions are prepared at a pH of between 6 and 8. In some aspects, the DDPA compositions have a pH of between 5 and 6 so as to match the natural skin pH. Thus, formulations preferably do not appreciably change the skin's natural pH balance.

Suitable buffers include an acid, an acid salt, or a mixture thereof. Suitably, the acid is selected from the group consisting of lactic acid, acetic acid, maleic acid, succinic acid, citric acid, benzoic acid, boric acid, sorbic acid, tartaric acid, edetic acid, phosphoric acid, nitric acid, sulphuric acid and hydrochloric acid, and mixtures thereof. In other aspects, the pH adjusting agent is a buffer. Suitably, the buffer is selected from the group consisting of citrate/citric acid, acetate/acetic acid, phosphate/phosphoric acid, propionate/propionic acid, lactate/lactic acid, ammonium/ammonia and edetate/edetic acid. In one aspect, the pH adjusting agent is a buffer which is citrate/citric acid.

In aspects provided herein, the pH adjusting agent is present in the composition in an amount from about 0.01% to about 10% by weight, based on the total weight of the composition. In an aspect, the pH of the composition is adjusted with a pH adjusting agent to a pH of from about 5 to about 8, such as from 6 to 7. In other aspects, the DDPA compositions are prepared as slightly acid formulations with a pH of between 5.5 and 7.

### Preservatives

The present topical pharmaceutical compositions of DDPA may further comprise a preservative. Preservatives are generally known in the art. In an aspect, the preservative is a mixture of two or more preservatives.

Exemplary preservatives include, but are not limited to, benzyl alcohol, imidazolidinyl urea, diazolidinyl urea, dichlorobenzyl alcohol, chloroxylenol, methyl paraben, ethyl paraben, propyl paraben, butyl paraben, phenoxyethanol, sorbic acid, benzoic acid, salts thereof, and mixtures thereof.

In an aspect, the preservative is selected from the group consisting of benzyl alcohol, phenoxyethanol and benzoic acid, and mixtures thereof.

In one aspect, the preservative is benzyl alcohol. In another aspect, the preservative is phenoxyethanol. In yet another aspect, the preservative is benzoic acid.

Suitably, the preservative is present in the composition in an amount from about 0.01% to about 2% by weight, based on the total weight of the composition. In one aspect, the preservative is present in the composition in an amount of about 0.25% by weight, based on the total weight of the composition.

### Penetration Enhancers

The present specification provides for, and includes, compositions of DDPA comprising penetrations enhancers. In aspects, emulsions of DDPA suitable for topical application further include a penetration enhancer. In an aspect, the penetration enhancer is a mixture of two or more penetration enhancers. The co-solvent or mixture of two or more co-solvents described herein may function as a penetration enhancer. Suitable penetration enhancers are known in the art, and include but are not limited to, fatty acids, fatty acid esters, fatty alcohols, pyrrolidones, sulfoxides, alcohols, diols and polyols, and mixtures thereof.

The present application provides for, and includes, penetration enhancer comprising 0.5% to 40% by weight of the composition. In aspects, the penetration enhancer comprises from about 1% to about 20% by weight. In other aspects, the penetration enhancer comprises about 5% to about 15% by weight, based on the total weight of the composition.

As provided herein, compositions of DDPA may include fatty acids as penetration enhancers that include, but are not limited to, oleic acid, capric acid, hexanoic acid, lauric acid, linoleic acid, linolenic acid, propionic acid and vaccenic acid, and mixtures thereof.

In aspects, exemplary fatty acid esters for use as penetration enhancers include, but are not limited to, glycerol monolaurate, glycerol monooleate, glycerol monolinoleate, isopropyl isostearate, isopropyl palmitate, isopropyl myristate, diethylsebacate, sorbitan monopalmitate, sorbitan oleate, sorbitan dilaurate, sorbitan trioleate, propylene glycol monolaurate and sucrose monolaurate, and mixtures thereof.

In further aspects, the present application provides for DDPA compositions having fatty alcohols as penetration enhancers. Suitable fatty alcohols include, but are not limited to, cetyl alcohol, stearyl alcohol, decanol, tridecanol, lauryl alcohol, linolenyl alcohol and oleyl alcohol, and mixtures thereof.

Other penetration enhancers suitable for use in the DDPA composition of the present application include pyrrolidones, such as, but not limited to, N-methyl pyrrolidone, 2-pyrrolidone and N-cyclohexyl-2-pyrrolidone, and mixtures thereof.

As provided herein, DDPA compositions may include as penetration enhancers a sulfoxide including, but are not limited to, dimethyl sulfoxide and decylmethyl sulfoxide, and mixtures thereof.

Alcohols are useful as penetration enhancers in the present DDPA compositions. Exemplary alcohols include, but are not limited to, lower (C1-C6) alcohols and diethylene glycol monoethyl ether, and mixtures thereof. Also included and provided for as penetration enhancers of the presently provided DDPA composition are diols or polyols. Exemplary diols include, but are not limited to, 1,2-hexanediol, butylene glycol, diethylene glycol, dipropylene glycol, ethyl hexanediol, ethylene glycol, hexylene glycol, pentylene glycol, propylene glycol, propylene glycol monolaurate, tetraethylene glycol, triethylene glycol, tripropylene glycol, polyethylene glycol and polypropylene glycol, and mixtures thereof. Exemplary polyols include, but are not limited to, butanetriol, glycerol and 1,2,6-hexanetriol, and mixtures thereof.

### Combinations of DDPA with Probiotics

In aspects, the compositions of DDPA further include an isolated Lactobacillus reuteri strain that does not produce DDPA. Lactobacillus reuteri strains that do not produce DDPA are generally strains that do not have the PKS locus. In an aspect, the isolated Lactobacillus reuteri strain that does not produce DDPA is Lactobacillus reuteri strain 3630, a representative sample of cells of the strain having been deposited with the ATTC under ATCC Accession No PTA-126787.

### Therapeutic Amount

The compositions of the present application include, and provide for, a therapeutic amount of DDPA. As shown in the examples, below, DDPA when presented in its natural pro-biotic form is unstable and lacks effectiveness for the treatment of allergy induced atopic dermatitis in dogs. Not to be limited by theory, it is believed that the lack of activity is due to the unstable nature of the DDPA, and low concentration found in its native form. Further, it was observed that the application of the DDPA expression probiotic led to the accumulation of solid materials over time on the skin. It is believed that this further reduces the penetration of the DDPA into the skin. In contrast, it is believed that DDPA expressing probiotics, when used in the gastrointestinal system, do not suffer from the same limitations. In the gut, there are solubilizing factors (e.g., bile salts) and transporters that facilitated absorption, the environment is moist and compatible with probiotic bacterial growth.

The identification of an active AhR agonizing component and determining its properties allows for the preparation of effective topical compositions that overcome the issues associated with probiotic application and the delivery of therapeutic amounts of DDPA. In an aspect, the DDPA or a pharmaceutically acceptable salt thereof is present in the composition in a therapeutically acceptable amount. In aspects, composition comprises DDPA in a therapeutically acceptable amount from about 0.01% to about 5% by weight. In other aspects, the composition comprises DDPA in a therapeutically acceptable amount about 0.05% to about 2% by weight, based on the total weight of the composition. In another aspect, the DDPA or a pharmaceutically acceptable salt thereof is present in an amount from about 0.1% to about 1.0% by weight, based on the total weight of the composition. In an aspect, the DDPA or a pharmaceutically acceptable salt thereof is present in an amount of about 0.25%, 0.30%, 0.40%, 0.50%, 0.75%, 1% or 2% by weight, based on the total weight of the composition. In one aspect, the DDPA or a pharmaceutically acceptable salt thereof is present in an amount of about 0.25% to about 0.50% by weight.

### Exemplary Topical Compositions of DDPA

The present application includes, and provides for, exemplary compositions for the topical delivery of DDPA and the treatment of inflammatory skin disorders

In an aspect, the topical DDPA composition comprises a cream formed from an emulsion of DDPA comprising 0.01% to about 5% by weight prepared as a hydrogel comprising a polymer, polyhydric alcohol, and water, wherein water and volatiles are present in an amount of less than 20% by weight and less than 50% hydrocarbons, waxes, or polyols as excipients.

In an aspect, the topical DDPA composition comprises a cream formed from an emulsion of DDPA comprising 0.01% to about 5% by weight prepared as an ointment formed from an emulsion of DDPA wherein the ointment comprises less than 20% water and volatiles, and greater than 50% hydrocarbons, waxes, or polyols, as excipients.

In an aspect, the the topical DDPA composition comprises an emulsion of DDPA, and a hydrogel formed from a polymer, polyhydric alcohol, and water, wherein water is present in an amount of about 50% to about 99% by weight.

### Dosage Forms

The DDPA pharmaceutical compositions can be used in the preparation of individual, single unit dosage forms or as bulk forms (e.g., tubes containing a cream, ointment, or lotion). Pharmaceutical compositions and dosage forms provided herein comprise DDPA, or a pharmaceutically acceptable salt, and one or more excipients. In aspects, the DDPA compositions and dosage forms provided herein can further comprise one or more additional active ingredients.

In aspects, the topical DDPA compositions can further comprise one or more active ingredients as a second active agent. In aspects, the second active agent is selected from dupilumab; crisaborole; calcineurin inhibitors, such as tacrolimus and pimecrolimus; antibiotics; hydrocortisone; corticosteriods, such as prednisone; antihistamines, such as cetirizine, fexofenadine, diphenhydramine; and combinations thereof. In aspects, the second active agent is selected from apremilast (Otezla™); adalimumab; secukinumab; guselkumab; ixekizumab; etanercept; infliximab; ustekinumab; golimumab; apremilast; topical corticosteriods (TCS), such as prednisone; vitamin D; vitamin D derivatives, such as calcipotriene or calcitriol; combination of calcipotriene and betamethasone dipropionate (Enstilar™) anthralin; methotrexate; cyclosporine; vitamin A; vitamin A derivatives, such as retinoids, tazarotene, or acitretin; calcineurin inhibitors, such as tacrolimus and pimecrolimus; thioguanine; hydroxyurea; salicylic acid; coal tar; and combinations thereof.

Single unit dosage forms provided herein are suitable for topical administration to the skin for the treatment of inflammatory skin disorders. Examples of dosage forms include, but are not limited to: gels, creams, and ointments; liquid dosage forms suitable mucosal or dermal administration to a patient, including suspensions (e.g., aqueous, or non-aqueous liquid suspensions, oil-in-water emulsions, or a water-in-oil liquid emulsions), solutions for dermal spray application, and foams for dermal application. These and other ways in which specific dosage forms are used will vary from one to another will be readily apparent to those skilled in the art. See, pharmacologic references, supra.

The present application provides dosage forms that provide DDPA at a therapeutic level for different times. That is, some topical compositions are prepared to provide fast acting. fast dissipating amounts of DDPA. In an aspect, the DDPA composition is provided as a photosensitive composition, typically a spray, to apply to an inflammatory skin condition for an acute treatment. In other aspects, the topical composition is prepared to provide DDPA over an extended period, for example for once daily use. Such preparations comprise a photoprotective composition, usually a gel, cream, or an ointment. In aspects, the composition is an emulsion. In aspects the emulsion is opaque, thereby providing a photoprotected form.

In aspects, the compositions include one or more photoprotective agents. DDPA is photo-labile and must be protected from light during preparation, packaging, and storage. Once in a stored, light impermeable container, the photoinactivation and loss of activity of DDPA can be modified by including a photoprotective agent. Opaque photoprotective agents such as zinc oxide, titanium dioxide and others would provide for longer stability of the DDPA. Suitable photoprotectants are discussed above.

The compositions of the present application may conveniently be presented in dosage unit form and may be prepared by any of the methods well known in the art of pharmacy. For example, the composition can be placed in an appropriate container, such as a squeeze-tube with a cap for dispensing ointments and creams, or a device for dispensing unit dosages of the formulation, such as a bottle or dropper that dispenses a controlled pre-determined dosage of the composition to a target area. In aspects, the target area is on the skin of an animal. In an aspect, the treatment area is the skin of a dog or human.

### Methods

The present application provides for, and includes, methods for treating inflammatory skin conditions including atopic dermatitis (AD), psoriasis (Ps), contact dermatitis, hidradenitis suppurativa (HS), pyoderma gangrenosum (PG), palmoplantar pustulosis (PPP), prurigo nodularis (PN), Sweet's syndrome, mutations in proline-serine-threonine phosphatase interacting protein 1 (PSTPIP1) (PAPA syndrome, PAPSH syndrome and PASH syndrome), Bechet's disease, bullous pemphigold, mucous membrane pemphigold, pemphis vulgaris, cutaneous Crohn's disease, Sjögren syndrome, systemic lupus erythematosus, pityriasis lichenoides chronica, and erythoderma. In aspects, the methods comprise applying a composition of the AhR agonist DDPA to the skin of an animal in need thereof, said composition comprising Dodeca-2,4,6,8,10-pentaenoic acid (DDPA) at a concentration of 0.01% to 5% (w/w) and one or more excipients selected from the group consisting of diluents, fillers, disintegrants, binders, lubricants, surfactants, hydrophobic vehicles, water soluble vehicles, emulsifiers, buffers, humectants, moisturizers, solubilizers, preservatives, colorants, plasticizers, carriers, excipients, or combinations thereof.

The present application provides for a method of treating an inflammatory skin disorder comprising providing an emulsion comprising DDPA at a concentration of 0.01% to 5% (w/w) prepared as a topical formulation. In aspects, the topical formulation is a spray, an ointment, a cream, a foam, or a gel. In aspects, the emulsion comprising DDPA is applied twice daily, once daily, once every other day; twice weekly; three times weekly, or once weekly

The present application further includes, and provides for, a method of reducing expression of one or more proinflammatory cytokines in the skin of the treated animal. In an aspect, the reduction of proinflammatory cytokines reduction in inflammatory symptoms in a subject having an inflammatory skin disease. In aspects, the production or expression of one or more proinflammatory cytokine is selected from IL-6, TNF-α, IFN-γ and IL-1, including IL-1β is reduced in the skin of the treated animal.

The present specification further includes methods for increasing the production or expression of one or more anti-inflammatory cytokine is increased in the skin of the treated animal. In an aspect, the methods provide for the application of a DDPA containing composition to the skin of a subject in need to increase the production or expression of one or more anti-inflammatory cytokine selected from IL-10 and IL-22.

In aspects, the present methods include methods for treating an inflammatory skin disease comprising applying a topical composition to the skin of an animal subject in need of treatment, where said topical composition comprises an effective amount of an emulsion of Dodeca-2,4,6,8,10-pentaenoic acid (DDPA) and a pharmaceutically acceptable carrier. In aspects, the inflammatory skin disease is selected from dermatitis, dermatitis, alopecia, and psoriasis, pyoderma gangrenosum, palmoplantar pustulosis, prurigo nodularis, or hidradenitis suppurativa. Also included and provided for are combination therapies comprising a topical composition of DDPA and a second active ingredient comprising one or more of an anti-inflammatory agent, molecule, or cytokine.

The methods of the present application include treating an animal for an inflammatory skin condition wherein the animal is selected from a farm animal, a food production animal, a domestic animal, or poultry. In an aspect, the animal is a human having an inflammatory skin condition. In other aspects, the animal is a non-human animal. In aspects the non-human animal is a horse, dog, or cat.

The present application also includes, and provides for, methods for modifying the expression of one or more proinflammatory cytokines or one or more anti-inflammatory cytokine in the skin of an animal in need thereof comprising administering to the animal a composition comprising effective amount of an emulsion of Dodeca-2,4,6,8,10-pentaenoic acid (DDPA) and a pharmaceutically acceptable carrier. In an aspect, the modifying is is increasing the production or expression of one or more anti-inflammatory cytokines. In certain aspects, the methods provide for the decreased production or expression of one or more proinflammatory cytokine selected from IL-6, TNF-α, IFN-γ and IL-1, including IL-1β, in the skin of an animal. In aspect, the production or expression of one or more anti-inflammatory cytokine selected from IL-10 and IL-22 is increased in the skin of an animal.

The methods of the present application include combination therapies to treat an inflammatory skin disease. In aspects, methods further comprise administering one or more of an anti-inflammatory agent, molecule, or cytokine to a subject or animal. In the present methods, the composition comprising an effective amount of an emulsion of Dodeca-2,4,6,8,10-pentaenoic acid (DDPA) and a pharmaceutically acceptable carrier is administered prior to, in conjunction with, and/or following one or more anti-inflammatory agent, molecule or cytokine, or immunomodulatory agent, molecule or cytokine. Suitable anti-inflammatory agents, molecules, or cytokines are selected from one or more anti-inflammatory, nonsteroidal anti-inflammatory drug (NSAID), steroid, biologic, antibiotic, or anti-diarrheal agents.

In one aspect, the compositions comprise a therapeutically effective amount of the AhR agonist. The amount of compound administered depends upon a variety of factors, including, for example, the severity of the inflammatory condition in the animal being treated, the mode of administration, the age and weight of the patient, the bioavailability of the particular active compound, etc. Determination of an effective dosage is well within the capabilities of those skilled in the art. A skilled practitioner will be able to determine the optimal dose for a particular individual. Effective dosages may be estimated initially from in vitro assays.

The compositions of the present specification are applied to the skin, in various aspects, at least once daily, or at least two, three or four times daily. In one aspect, the topical formulation is applied at least once daily for a period of about 4-12 weeks. In other aspects, the topical formulation is applied between 1-3 times daily for a period between about 4-12 weeks. In other aspects, the composition is topically applied 1-2, 1-3, 1-4 or 1-5 times daily for a period of between about 1-2 weeks, 1-3 weeks, 1-4 weeks, 1-6 weeks, 1-8 weeks, 1-10 weeks, 1-12 weeks, 1-16 weeks, 1-20 weeks, 1-24 weeks, 1-2 months, 1-3 months, 1-4 months, 1-6 months, 1-8 months, 1-10 months, 1-12 months. The dosing schedule will depend on factors well known in medical arts, including the dose of drug compound in the formulation, the severity of the disorder, and the health of the patient.

### Preparation of DDPA Containing Emulsions

In aspects, the DDPA containing compositions are prepared as emulsions. As discussed above, emulsions provided for both solubilization of the hydrophobic DDPA compound and for protection of the DDPA from degradation. Suitable agents for preparing emulsions of DDPA are provided in Table 1.

TABLE 1
Agents for the preparation of DDPA emulsions

| Chemical name | HLB | Miscible with H2O | Function |
|---|---|---|---|
| Oleic acid | 1.6 | Immiscible | Antifoam |
| Sorbitan tristearate | 2.1 | Immiscible | Antifoam |
| Ethylene glycol | 2.9 | Immiscible | Antifoam |
| monostearate | | | |
| Glyceryl monostearate | 3.8 | Disperses with | W/O emulsifier |
| | | difficulty | |
| Sorbitan monostearate | 4.7 | Disperses with | W/O emulsifier |
| | | difficulty | |
| Sorbitan monopalmitate | 6.7 | Forms milky | W/O emulsifier |
| | | dispersion | |
| PEG-4 dilaurate | 6 | Forms milky | W/O emulsifier |
| | | dispersion | |
| Sucrose dipalmitate | 7.4 | Forms milky | W/O emulsifier |
| | | dispersion | |
| PEG-4 monooleate | 8 | Forms milky | W/O emulsifier |
| | | dispersion | |
| PEG-4 monolaurate | 9.8 | Form milky stable | O/W emulsifier |
| | | dispersion | |
| Polysorbate 85 | 11 | Form clear dispersion | O/W emulsifier |
| PEG-8 monooleate | 11.4 | Form clear dispersion | O/W emulsifier |

Emulsions are generally characterized by the drop size. Since emulsification is generally a more or less random stirring process in which the breaking and coalescence steps are in dynamic equilibrium, the resulting emulsion is a polydispersed system in which small and big drops coexist. The outcome of this equilibrium depends upon a large number of factors that can influence the two antagonistic steps. Generally speaking, a lower tension or an increase in stirring energy and duration are expected to increase the breakup, while a higher fluid viscosity would slow it down, and temperature would increase the coalescence rate (3-7).

The emulsions comprising DDPA can comprise a drop distribution of different types. In an aspect, the DDPA emulsion is a unimodal dispersion of the lognormal type produced by turbulent homogeneous stirring. In an aspect, the DDPA emulsion droplet distribution is narrowly shaped or highly polydispersed. Also included and provided for are bimodal emulsions resulting from a mixture of two emulsions, for example to attain a low viscosity.

As used herein, the term “D90” refers to the oil droplet size diameter of which 90% of the droplets are less than a particular size. Alternatively, the term “D90” is defined as the size in microns below which 90 percent of the oil droplets reside on a volume basis. In an aspect, the D90 of the average droplet size of the discontinuous phase in the composition is less than 15 microns. In another aspect, the average droplet size of the discontinuous phase according to the present invention has a D90 of less than 5 microns. In aspects, the DDPA containing emulsions have a D90 of less than 15.

As used herein, the term “D50” refers to the median or 50th percentile of which the

droplets are less than a particular size. Alternatively, the term “D50” is defined as the size in microns below which 50 percent of the oil droplets reside on a volume basis. In an aspect, the compositions comprising and emulsion of a therapeutic amount of DDPA have a D50 of the average droplet size of the discontinuous phase in the composition of less than 5 microns. In another aspect, the average droplet size of the discontinuous phase of the DDPA containing emulsions according to the present application have a D50 of less than 1 micron.

Methods for measuring oil droplet size distribution are well known in the art. In one aspect, oil droplet size diameter distribution oil droplet size in the composition according to the present invention may be measured using laser diffraction techniques. Suitable laser diffraction apparatus includes, for example, the Sympatec HELOS/QUIXEL, or the Malvern Laser Diffractonamer obtainable from Malvern Instruments, Malvern, UK as well as others.

### Definitions

As used herein, “isolated” means that the subject isolate has been separated from at least one of the materials with which it is associated in a particular environment, for example, its natural environment.

As used herein, “about” means±10%.

As used herein, “probiotic” refers to a substantially pure microbe (i.e., a single isolate) or a mixture of desired microbes, and may also include any additional components (e.g., carrier) that can be administered to an animal to provide a beneficial health effect. Probiotics or microbial compositions of the invention may be administered with an agent or carrier to allow the microbes to survive the environment of the gastrointestinal tract, i.e., to resist low pH and to grow in the gastrointestinal environment.

Suitable excipients can be, without limitation, any adjuvant, carrier, excipient, glidant, sweetening agent, diluent, preservative, dye/colorant, flavor enhancer, fragrance, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

Suitable excipients can be sterile liquids, such as water and oils, including those of petroleum, animal, vegetable, or synthetic origin; such as peanut oil, soybean oil, mineral oil, sesame oil, and the like. The choice of carrier can be selected with regard to the intended topical route of administration and standard pharmaceutical practice.

Other suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, water, ethanol, and the like. The pharmaceutical composition, if desired, can also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MWS (2-(N-morpholino) ethanesulfonic acid), or can contain detergents, like Tween, poloxamers, poloxamines, CHAPS, Igepal, or amino acids like, for example, glycine, lysine, or histidine. In some aspects, the excipient is selected from celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum, acacia (gum arabic), agar, aluminum magnesium silicate, sodium alginate, sodium stearate bladderwrack, bentonite, carbomer, carrageenan, carbopol, xanthan, cellulose, microcrystalline cellulose (MCC), ceratonia, chondrus, dextrose, furcellaran, gelatin, ghatti gum, guar gum, hectorite, lactose, sucrose, maltodextrin, mannitol, sorbitol, honey, maize starch, wheat starch, rice starch, potato starch, gelatin, sterculia gum, polyethylene glycol (e.g. PEG 200-4500), gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), or combinations thereof. In some aspects, the excipient is not a polysiloxane.

As used herein, “emulsion” describes a dosage form consisting of a two-phase system comprised of at least two immiscible liquids, one of which is dispersed as droplets (internal or dispersed phase) within the other liquid (external or continuous phase) generally stabilized with one or more emulsifying agents (US FDA Drug Nomenclature Monograph, number C-DRG-00201). Emulsions include creams, lotions, liniments, and ointments. Oil-in-water emulsions are colloidal dispersions system in which liquid oil is dispersed as droplets (the discrete phase, also referred to as “the discontinuous non-aqueous phase” or “the discontinuous phase”) in a continuous aqueous medium (the continuous phase, also referred to as “the continuous aqueous phase” or “the continuous phase”). In some aspects, at least 50% of the active ingredient (w/w) is dissolved and remains in the emulsion. In some aspects, at least 75% of the active ingredient (w/w) is dissolved and remains in the emulsion. In other aspects, as is further described herein, greater than 85% of the active ingredient is present in the discontinuous phase.

As used herein, a “lotion” is a liquid emulsion for external application to the skin. A liquid is pourable; it flows and conforms to its container at room temperature. It displays Newtonian or pseudoplastic flow behavior.

As used herein, “liniment” means a solution or mixture of various substances in oil, alcoholic solutions of soap, or emulsions intended for external application.

As used herein, “cream” means an emulsion in semisolid dosage form, usually containing >20% water and volatiles5 and/or <50% hydrocarbons, waxes, or polyols as the vehicle. This dosage form is generally for external application to the skin or mucous membranes. A semisolid is not pourable; it does not flow or conform to its container at room temperature. It does not flow at low shear stress and generally exhibits plastic flow behavior.

As used herein, an “ointment” is a semisolid dosage form, usually containing <20% water and volatiles and >50% hydrocarbons, waxes, or polyols as the vehicle. This dosage form is generally for external application to the skin or mucous membranes.

As used herein, a “patch” is a drug delivery system that often contains an adhesive backing that is usually applied to an external site on the body. Its ingredients either passively diffuse from, or are actively transported from, some portion of the patch. The ingredients are delivered to the outer surface of the body. A patch is sometimes synonymous with the terms extended-release film' and ‘system’.

As used herein, a “gelling agent” is an excipient that serves to increase the thickness or viscosity of the composition. In aspects, solutions are low viscosity formulations most typically without a gelling agent. Lotions, liniments, creams, and ointments are compositions with increasing amounts of a gelling agent.

As used herein, “delivery” or “administration” means the act of providing a beneficial activity, here DDPA, to an animal. The delivery may be direct or indirect. An administration could be by topical application of a composition to the skin, the nasal cavity, or a mucosal membrane. For example, without limitation, a nasal route of administration may be through a spray or vapor, a mucosal route of administration may be through direct contact with mucosal tissue, and topical route may be through a cream or ointment either applied directly or as a patch. Suitable mucosal tissues for administration of the DDPA compositions are tissues that are membrane rich in mucous glands such as those that line the inside surface of the nose, mouth, and anus.

As used herein, the terms “treating”, “to treat”, or “treatment”, include restraining, slowing, stopping, reducing, ameliorating, or reversing the progression or severity of an existing symptom, disorder, condition, or disease. A treatment may also be applied prophylactically to prevent or reduce the incidence, occurrence, risk, or severity of a clinical symptom, disorder, condition, or disease.

As used herein, “subject” includes animals having an inflammatory skin condition. Suitable animals include farm animals, food production animals, domestic animals, or poultry. In aspects, the subject is a human, horse, dog, or cat. bird, poultry, a human, or a non-human mammal. Specific food productions animals include chickens, turkey, dogs, cats, cattle, and swine. The chicken may be a broiler chicken, egg-laying, or egg-producing chicken. As used herein, the term “poultry” includes domestic fowl, such as chickens, turkeys, ducks, quail, and geese.

By “stimulating” is meant directly or indirectly increasing the level and/or functional activity of a target system (e.g., immune system). In certain aspects, “stimulation” or “stimulating” means that a desired/selected response is more efficient (e.g., at least 10%, 20%, 30%, 40%, 50%, 60% or more), more rapid (e.g., at least 10%, 20%, 30%, 40%, 50%, 60% or more), greater in magnitude (e.g., at least 10%, 20%, 30%, 40%, 50%, 60% or more), and/or more easily induced (e.g., at least 10%, 20%, 30%, 40%, 50%, 60% or more) than if the vaccine had been used alone,

### Embodiments

Embodiment 1: A composition comprising Dodeca-2,4,6,8,10-pentaenoic acid (DDPA) and an at least one excipient selected from the group consisting of diluents, fillers, disintegrants, binders, lubricants, surfactants, hydrophobic vehicles, water soluble vehicles, co-solvents, emulsifiers, buffers, humectants, moisturizers, solubilizers, preservatives, colorants, plasticizers, carriers, penetration enhancers, or combinations thereof.
Embodiment 2: The composition of embodiment 1, wherein said composition is an emulsion.
Embodiment 3: The composition of any one of embodiments 1 or 2, wherein said emulsion of DDPA is a topical formulation for application to the skin of an animal, said topical formulation comprising a spray, an ointment, a cream, a foam, or a gel.
Embodiment 4: The composition of any one of embodiments 1 to 3, wherein said topical formulation is for use in the preparation of a medication for the treatment of an inflammatory skin condition.
Embodiment 5: The composition of any one of embodiments 1 to 4, wherein said DDPA comprises a therapeutically effective amount.
Embodiment 6: The composition of any one of embodiments 1 to 5, wherein said DDPA comprises 0.01% to about 5% by weight of said composition.
Embodiment 7: The composition of any one of embodiments 2 to 6, wherein said emulsion comprises an oil phase, a water phase, and a surfactant.
Embodiment 8: The composition of any one of embodiments 2 to 6, wherein said emulsion is self-emulsifying.
Embodiment 9: The composition of any one of embodiments 2 to 8, said composition is an oil-in-water emulsion.
Embodiment 10: The composition of any one of embodiments 2 to 8, wherein said composition is a water-in-oil emulsion.
Embodiment 11: The composition of any one of embodiments 1 to 10, wherein the pH of said composition is between 6 and 8.
Embodiment 12: The composition of any one of embodiments 1 to 10, wherein said excipients are acceptable pharmaceutically, cosmetically, or both.
Embodiment 13: The composition of any one of embodiments 1 to 12, wherein said excipient comprises an antioxidant, a preservative, a penetration enhancer, a chelating agent, pH adjusting agent, a humectant, a gelling agent, a fragrance, a colorant, and combinations thereof.
Embodiment 14: The composition of any one of embodiments 1 to 13, wherein said composition comprises a light protective excipient.
Embodiment 15: The composition of any one of embodiments 1 to 14, wherein said composition is a homogenous emulsion.
Embodiment 16: The composition of any one of embodiments 2 to 15, wherein said emulsion comprises average oil droplet size of less than 35 microns.
Embodiment 17: The composition of any one of embodiments 2 to 16, wherein said composition comprises said emulsion of DDPA and a hydrogel formed from a polymer, polyhydric alcohol, and water, wherein water is present in an amount of about 50% to about 99% by weight.
Embodiment 18: The composition of any one of embodiments 2 to 16, wherein said composition comprises a cream formed from said emulsion of DDPA and comprises a hydrogel comprising a polymer, polyhydric alcohol, and water, wherein water and volatiles are present in an amount of less than 20% by weight and less than 50% hydrocarbons, waxes, or polyols as excipients.
Embodiment 19: The composition of any one of embodiments 2 to 16, wherein said composition comprises an ointment formed from said emulsion of DDPA wherein said ointment comprises less than 20% water and volatiles, and greater than 50% hydrocarbons, waxes, or polyols, as excipients.
Embodiment 20: The composition of any one of embodiments 1 to 19, further comprising an isolated Lactobacillus reuteri strain that does not produce DDPA.
Embodiment 21: The composition of embodiment 20, wherein said isolated Lactobacillus reuteri strain that does not produce DDPA is Lactobacillus reuteri strain 3630, a representative sample of cells of said strain having been deposited with the ATTC under ATCC Accession No PTA-126787.
Embodiment 22: A method of treating an inflammatory skin condition in animal in need thereof comprising applying a composition comprising an emulsion of Dodeca-2,4,6,8,10-pentaenoic acid (DDPA) and one or more excipients selected from the group consisting of diluents, fillers, disintegrants, binders, lubricants, surfactants, hydrophobic vehicles, water soluble vehicles, emulsifiers, buffers, humectants, moisturizers, solubilizers, preservatives, colorants, plasticizers, carriers, excipients, or combinations thereof.
Embodiment 23: The method of embodiment 22, wherein said condition comprises atopic dermatitis (AD), psoriasis (Ps), contact dermatitis, hidradenitis suppurativa (HS), pyoderma gangrenosum (PG), palmoplantar pustulosis (PPP), prurigo nodularis (PN), Sweet's syndrome, mutations in proline-serine-threonine phosphatase interacting protein 1 (PSTPIP1) (PAPA syndrome, PAPSH syndrome and PASH syndrome), Bechet's disease, bullous pemphigold, mucous membrane pemphigold, pemphis vulgaris, cutaneous Crohn's disease, Sjögren syndrome, systemic lupus erythematosus, pityriasis lichenoides chronica, and erythoderma.
Embodiment 24: The method of any one of embodiments 22 or 23, wherein said composition is applied twice daily, once daily, once every other day; twice weekly; three times weekly, or once weekly.
Embodiment 25: The method of any one of embodiments 22 to 24, wherein the production or expression of one or more proinflammatory cytokine in the skin of the treated animal is reduced.
Embodiment 26: The method of any one of embodiments 22 to 25, wherein said production or expression of one or more proinflammatory cytokine selected from IL-6, TNF-α, IFN-γ and IL-1, including IL-1B, is reduced in the skin of a treated animal.
Embodiment 27; The method of any one of embodiments 22 to 26, wherein said production or expression of one or more anti-inflammatory cytokine is increased in the skin of the treated animal.
Embodiment 28: The method of any one of embodiments 22 to 27, wherein said production or expression of one or more anti-inflammatory cytokine selected from IL-10 and IL-22 is increased in the skin of the treated animal.
Embodiment 29: A method for treating an inflammatory skin disease comprising applying a topical composition to the skin of an animal subject in need of treatment, wherein said topical composition comprises an effective amount of an emulsion of Dodeca-2,4,6,8,10-pentaenoic acid (DDPA) and a pharmaceutically acceptable carrier.
Embodiment 30: The method of embodiment 29, wherein said inflammatory skin disease is selected from dermatitis, dermatitis, alopecia, and psoriasis, pyoderma gangrenosum, palmoplantar pustulosis, prurigo nodularis, or hidradenitis suppurativa.
Embodiment 31: The method of any one of embodiments 29 or 30, wherein said composition further comprises administering one or more of an anti-inflammatory agent, molecule, or cytokine.
Embodiment 32: The method of any one of embodiments 29 to 31, wherein the animal is a farm animal, a food production animal, a domestic animal, or poultry.
Embodiment 33: The method of any one of embodiments 29 to 32, wherein the animal is a human.
Embodiment 34: The method of any one of embodiments 29 to 32, wherein the animal is a horse, dog, or cat.
Embodiment 35: A method for modifying the expression of one or more proinflammatory cytokines or one or more anti-inflammatory cytokine in the skin of an animal in need thereof comprising administering to the animal a composition comprising effective amount of an emulsion of Dodeca-2,4,6,8,10-pentaenoic acid (DDPA) and a pharmaceutically acceptable carrier.
Embodiment 36: The method of embodiment 35, wherein said modifying is reducing the production or expression of one or more proinflammatory cytokines.
Embodiment 37: The method of any one of embodiments 35 or 36, wherein said modifying is increasing the production or expression of one or more anti-inflammatory cytokine.
Embodiment 38: The method of any one of embodiments 35 to 37, wherein the production or expression of one or more proinflammatory cytokine selected from IL-6, TNF-α, IFN-γ and IL-1, including IL-1B, is decreased in the skin of the animal.
Embodiment 39: The method of any one of embodiments 35 to 38, wherein the production or expression of one or more anti-inflammatory cytokine selected from IL-10 and IL-22 is increased in the skin of the animal.
Embodiment 40: The method of any one of embodiments 35 to 39, wherein said animal in need thereof has an inflammatory skin disease selected from dermatitis, dermatitis, alopecia, and psoriasis, pyoderma gangrenosum, palmoplantar pustulosis, prurigo nodularis, or hidradenitis suppurativa.
Embodiment 41: The method of any one of embodiments 35 to 40, comprising further administering one or more of an anti-inflammatory agent, molecule or cytokine to the subject or animal.
Embodiment 42: The method of any one of embodiments 35 to 41, wherein the composition comprising an effective amount of an emulsion of Dodeca-2,4,6,8,10-pentaenoic acid (DDPA) and a pharmaceutically acceptable carrier is administered prior to, in conjunction with, and/or following one or more anti-inflammatory agent, molecule, or cytokine, or immunomodulatory agent, molecule or cytokine.
Embodiment 43: The method of embodiment 42, wherein the one or more anti-inflammatory agent, molecule or cytokine is selected from one or more anti-inflammatory, nonsteroidal anti-inflammatory drug (NSAID), steroid, biologic, antibiotic, or anti-diarrheal agent.
Embodiment 44: A composition for topical use in treating an inflammatory skin condition comprising an emulsion of Dodeca-2,4,6,8,10-pentaenoic acid (DDPA) and one or more excipients selected from the group consisting of diluents, fillers, disintegrants, binders, lubricants, surfactants, hydrophobic vehicles, water soluble vehicles, emulsifiers, buffers, humectants, moisturizers, solubilizers, preservatives, colorants, plasticizers, carriers, excipients, or combinations thereof.
Embodiment 45: The composition for topical use of embodiment 44, wherein said composition is an ointment, a cream, a foam, a gel, or a spray.
Embodiment 46: The composition for topical use of any one of embodiments 44 or 45, wherein said composition is an oil-in-water emulsion.
Embodiment 47: The composition for topical use of any one of embodiments 44 to 46, wherein said composition is a water-in-oil emulsion.
Embodiment 48: The composition for topical use of any one of embodiments 44 to 47 wherein said ointment, cream, foam, or gel is hydrophobic or lipophilic.
Embodiment 49: The composition for topical use of any one of embodiments 44 to 48, wherein said ointment, cream, foam, or gel is applied to a patch.
Embodiment 50: The composition for topical use of any one of embodiments 44 to 49, wherein said inflammatory skin disease is selected from dermatitis, dermatitis, alopecia, and psoriasis, pyoderma gangrenosum, palmoplantar pustulosis, prurigo nodularis, or hidradenitis suppurativa.
Embodiment 51: The composition for topical use of any one of embodiments 44 to 50, wherein said inflammatory disease is selected from the group consisting of atopic dermatitis, seborrheic dermatitis, and psoriasis.
Embodiment 52: The composition for topical use of any one of embodiments 44 to 51, wherein said composition comprises a light protective component.
Embodiment 53: The composition for topical use of any one of embodiments 52, wherein said light protective component comprises zinc oxide or titanium oxide.
Embodiment 54: The composition for topical use of any one of embodiments 44 to 53, wherein said composition is opaque.
Embodiment 55: The composition for use of any one of embodiments 44 to 54, further comprising an isolated Lactobacillus reuteri strain that does not produce DDPA.
Embodiment 56: The composition for use of embodiment 55, wherein said isolated Lactobacillus reuteri strain that does not produce DDPA is Lactobacillus reuteri strain 3630, which corresponds to ATCC Patent Deposit Number PTA-126787.
Embodiment 57: The composition for use of any one of embodiments 44 to 56, wherein the production or expression of one or more proinflammatory cytokine in the skin of the animal is reduced.
Embodiment 58: The composition for use of embodiment 57, wherein the production or expression of one or more proinflammatory cytokine that is reduced selected from IL-6, TNF-α, IFN-γ and IL-1, including IL-1B, is reduced in the skin system of the animal.
Embodiment 59: The composition for use of any one of embodiments 44 to 58, wherein the production or expression of one or more anti-inflammatory cytokine is increased in the skin of the animal.
Embodiment 60: The composition for use of embodiment 59, wherein the production or expression of one or more anti-inflammatory cytokine selected that is increased is IL-10, IL-22, or both, is increased in the skin of the animal.
Embodiment 61: The composition for use of any one of embodiments 44 to 60, wherein the animal is bird, poultry, a human, or a non-human mammal.
Embodiment 62: The composition for use of embodiment 61, wherein the animal is a farm animal or food production animal, a domestic animal, or wherein the bird is poultry.
Embodiment 63: The composition for use of embodiment 61, wherein the animal is a human.
Embodiment 64: The composition for use of embodiments 61, wherein the animal is a horse. dog, or cat.

Any examples or illustrations given herein are not to be regarded in any way as restrictions on, limits to, or express definitions of any term or terms with which they are utilized. Instead, these examples or illustrations are to be regarded as being described with respect to one particular aspect and as being illustrative only. Those of ordinary skill in the art will appreciate that any term or terms with which these examples or illustrations are utilized will encompass other aspects which may or may not be given therewith or elsewhere in the specification and all such aspects are intended to be included within the scope of that term or terms. Language designating such nonlimiting examples and illustrations includes, but is not limited to: “for example,” “for instance,” “e.g.,” and “in one aspect.” In this specification, groups of various parameters containing multiple members are described. Within a group of parameters, each member may be combined with any one or more of the other members to make additional sub-groups. For example, if the members of a group are a, b, c, d, and e, additional sub-groups specifically contemplated include any one, two, three, or four of the members, e.g., a and c; a, d, and e; b, c, d, and e; etc.

### EXAMPLES

### Example 1: AhR Activity of L. reuteri 3632 and 3630 Whole Cells, Cell-Free Supernatants and Purified PKS Metabolite from L. reuteri 3632

An AhR activity assay is performed using HepG2-Lucia™ AhR reporter cells (Invivogen) following manufacturer's instructions. The AhR agonist 6-Formylindolo[3,2-b]carbazole (FICZ, 1 μg/ml final concentration) is used as a positive control. The test articles included L. reuteri 3632 and 3630 whole cells and cell-free supernatants and purified PKS metabolite [50 μg/ml in phosphate buffered saline (PBS)]. For preparing whole cells, overnight grown L. reuteri 3632 and 3630 are centrifuged at 10,000 rpm for 10 min at 4° C.; the supernatants are filter sterilized with 0.22 μM filter, the whole cells are resuspended in 10 ml of PBS. The media control, whole cells and cell-free supernatant samples are further diluted seven-fold in PBS and twenty microliters of the sample is mixed with 180 μl of cells containing 1.5×10⁵ cells/ml in a flat-bottom 96-well plate and incubated for 36 h in a CO₂ incubator. Following 36 h of incubation, 20 μl of the sample is mixed with 50 μl of Quanti-Luc™ and luciferase activity is measured immediately. The RLUs for whole cells, cell-free supernatants and purified PKS metabolite are normalized to either MRS media or PBS. The results are shown in Figure x. The data represents mean±standard deviation of 3 independent experiments. ***P<0.001.

### Example 2: Effects of Probiotic on House Dust Mite Induced Atopic Dermatitis in Allergic Dogs

The clinical response to topical post-/probiotic as preventative following challenge with house dust mite (HDM) allergens in dogs is assessed. Dogs are selected from an HDM allergic colony based upon their lesion scores change from baseline during the last time that they were challenged with HDM, Twenty-four dogs with a change in lesion score ³10 are included in this study

The 24 dogs are randomly divided into 4 treatments groups (i.e. Placebo, Cortavance, Treatments A and B), 3 incomplete crossover challenge phases, and 12 unique sequences (1-12) shown in Table 2 below. The first phase of the HDM challenges begins on Day 0, and the second and third phases should begin on Days 35 and 70, respectively. The person(s) administering the test article are unblinded. Personnel evaluating skin lesions are blinded to treatment. The study design will be composed of mutually orthogonal Latin squares with characteristics of a generalized Youden design that also balances for first-order carryover effects.

TABLE 2
Randomization of treatments

| | Challenge Period (treatment group) | | | Animal |
|---|---|---|---|---|
| Sequence | Phase I | Phase II | Phase III | ID |
| 1 | Placebo | Cortavance | Treatment A | 18 |
| 2 | Cortavance | Placebo | Treatment B | 6 |
| 3 | Treatment A | Treatment B | Placebo | 15 |
| 4 | Treatment B | Treatment A | Cortavance | 2 |
| 5 | Placebo | Treatment A | Treatment B | 17 |
| 6 | Cortavance | Treatment B | Treatment A | 14 |
| 7 | Treatment A | Placebo | Cortavance | 10 |
| 8 | Treatment B | Cortavance | Placebo | 7 |
| 9 | Placebo | Treatment B | Cortavance | 12 |
| 10 | Cortavance | Treatment A | Placebo | 22 |
| 11 | Treatment A | Cortavance | Treatment B | 16 |
| 12 | Treatment B | Placebo | Treatment A | 23 |

Topical treatments are applied to dogs while they are restrained in dorsal recumbency in a foam “V” trough. All dogs in each treatment are treated before moving to the next treatment. Treatments are as follows, and occur in the following order each day:
1. Placebo—Dogs receive topical spray containing placebo solution at the same timepoints as treatments A and B.
2. Cortavance™—Positive control. Dogs are sprayed over the abdomen and thorax (over the HDM challenge site) until the skin is wet. This will require multiple sprays. The number of ‘pumps’ from the sprayer that are required to cover the area are recorded and this same amount is used in subsequent treatments. After skin treatment, the test article is allowed to absorb into the skin for two minutes, and then the dog is returned to its cage.
3. Treatment A—The test article is a water-based spray formulation containing heatkilled bacteria. Persons handling animals wear gloves and change the gloves between treatment groups. All dogs are handled by treatments to avoid cross contamination. The test article is sprayed into the skin of the challenge area and rubbed lightly until the material has contacted all of the skin. Full coverage may require more than one spray. The spray amount required to cover the challenge area is recorded and the same amount is used for subsequent treatments. Similar amounts are applied for all dogs with adjustments made for larger or smaller dogs.
4. Treatment B—same as treatment A but using live probiotic strain.

All dogs are acclimated to the study site for at least 7 days prior to study activities. During this time, the dogs are acclimated to a 12 hour/12 hour light/dark cycle. On Day-7, dogs are hair clipped on the abdomen, thorax, and inside of the legs to allow better contact with the HDM.

For dogs receiving placebo, Treatments A and B, on the morning of Day-5, dogs have skin lesion scoring, followed by topical probiotic application (according to site SOPs). The topical application is repeated in the afternoon. This treatment is repeated through Day 3.

On the morning of Day 0, all dogs have skin lesion scoring, followed by HDM challenge (according to site SOPs). For dogs receiving placebo, Cortavance, treatments A, or B, these topical applications are given 1 hour prior to HDM challenge.

Skin lesion scoring continues through Day 14. HDM challenge continues through Day 2. Topical treatment with placebo, Cortavance, and topical post-/probiotics (Treatment A and B) continues through Day 3.

The dogs used in this study are sensitized to HDM and develop clinical skin lesions, primarily erythema and papules, when challenged with a topical application of crushed, whole bodied Dermatophagoides farinae (HDM). All HDM challenges are conducted according to Ridglan Farms site SOP for HDM challenges. (RDG-SOP-RES-700-38)

House Dust Mites obtained from Greer Laboratories, Lenoir, NC, USA is used as the challenge material. The HDM solution is prepared from HDM powder and mixed with PBS (pH=7.2) to obtain the target final concentration of 6.25 mg/mL. The HDM solution is prepared fresh on each day of application and sonicated for at least 15 minutes prior to use.

Immediately prior to the first HDM application of each challenge (i.e. day 0, day 35, day 70), the clipped area of the dog's skin (medial thighs, inguinal region, ventral abdomen, ventral thorax, and axillae) is stripped using sticky tape. Tape stripping removes part of the stratum corneum to ensure better penetration of the HDM allergen into the epidermis. The HDM solution in PBS is applied directly to the dog's tape-stripped skin by spraying approximately 8 mL (50 mg/dog) of the preparation. Dogs are challenged with HDM on three consecutive days in the morning. There are three HDM challenges in this study, which commence approximately on Days 0, 35, and 70.

Skin swabs, fecal samples, blood samples, and room samples are collected using standard methods.

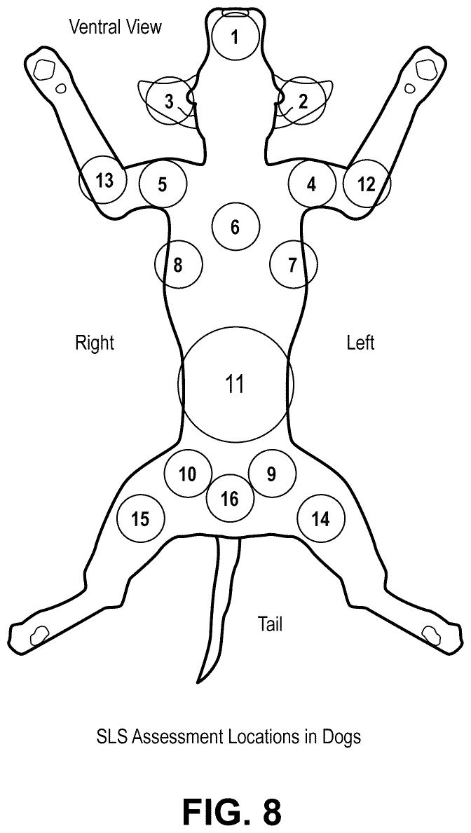

Lesion scoring: Dogs are evaluated by a trained person using the Skin Lesion Score data collection form. The lesion scoring is conducted by the same person throughout the study, where possible, to ensure consistency of the scoring. The SLS (modified from the CADESI-4) is be used throughout this study. It involves evaluation of 16 specific body sites (FIG. 8).

The total daily lesion score is calculated by adding the scores of all clinical signs and body sites.

Each body site is scored according to the following clinical signs: Erythema (redness/inflammation); Papules (raised area of skin without discharge); Excoriation (skin rubbing) and graded from 0 to 3: 0=not present; 1=mild; 2=moderate; 3=severe.

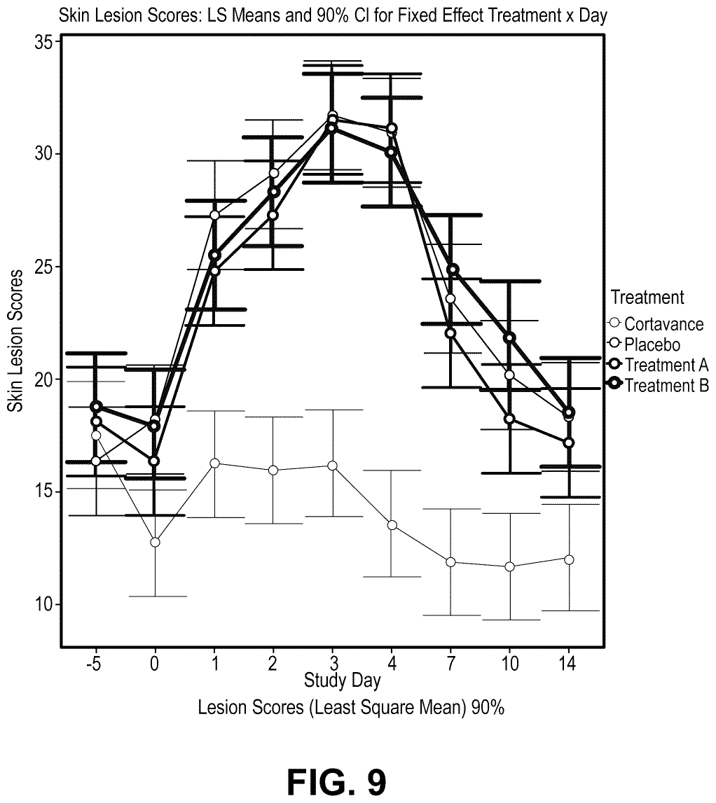

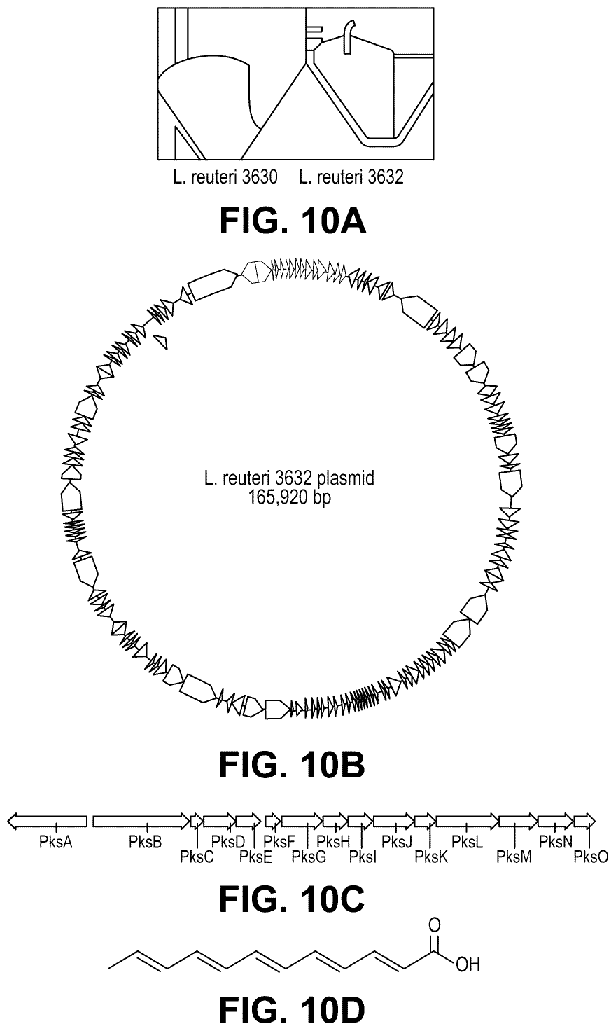

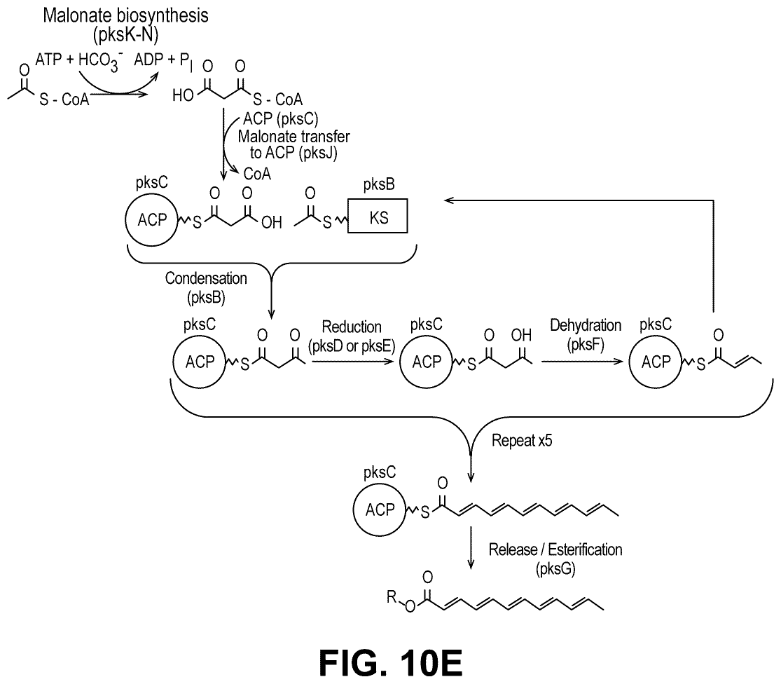

Results: No significant effects of topical administrations of AOM (postbiotic) on the improvement of SLS following HDM challenge in canine AD model are observed (FIG. 9). Similarly, no significant effect observed following topical administrations of live L. reuteri 3632 in HDM CAD animals. However, testing reveals that L. reuteri 3632 does not survive for a long time on the skin and postbiotic application could be considered. The study does not rule out a possibility of effects of L. reuteri due to the lack of survivability and the accumulation of solid materials over time for Treatments A and B.

### Example 3: Improving Expression of DDPA in Cultured Cells

To increase the yield of DDPA in cultured L. reuteri 3632 cells, an improved media is developed.

Starter cultures are grown in a BactoBox @39° C. for 16-17 hours prior to culture. Cultures are grown in ULCM media (Table 3) or ULCM Enrichment media (Table 4) in 90% nitrogen and 10% CO₂, at 39° C., with 100 RPM agitation and maintained at a pH of 5.5 with a dead band of 0.05. Cells from the starter culture are inoculated at a 5% rate and grown for 5.5 hours (e.g., stationary phase).

TABLE 3
ULCM Media

| Ingredient | Amount (g/L) | |
|---|---|---|
| Water | 800 | |
| K₂HPO₄ | 4 | |
| MgSO₄•7H20 | 0.1 | |
| MnSO₄•H₂0 | 0.05 | |
| Citric acid diammonium salt | 2 | |
| Yeast Extract (BD) | 10 | |
| FeSO₄•7H₂0 | 0.2 | |
| Betaine | 0.03 | |
| Sucrose | 50 | |
| A2 Stock solution | 2 | ml |
| Water | QS to 1000 ml | |
| PPG 2000 (sterile filtered) | 0.5-1.0 | ml |
| Dissolve components, adjust pH to 6.5 +/− 0,2 | | |
| with NaOH, filter sterilize | | |

TABLE 4
ULCM Enrichment media

| Ingredient | Amount (g/L) | |
|---|---|---|
| Water | 800 | |
| K₂HPO₄ | 2 | |
| MgSO₄•7H20 | 0.1 | |
| MnSO₄•H20 | 0.066 | |
| Citric acid diammonium salt | 2 | |
| Yeast Extract (BD) | 10 | |
| Sucrose | 50 | |
| Tween 80 | | |
| Water | QS to 1000 ml | |
| A2 Stock solution | 2 | ml |
| PPG 2000 (sterile filtered) | 0.5-1.0 | ml |
| Dissolve components, adjust pH to 6.5 +/− 0.2 | | |
| with NaOH, filter sterilize | | |

### Example 4: Effects of DDPA Compositions on House Dust Mite Induced Atopic Dermatitis in Allergic Dogs

The clinical response to topical DDPA compositions as preventative following challenge with house dust mite (HDM) allergens in dogs is assessed. Dogs are selected from an HDM allergic colony based upon their lesion score change from baseline during the last time that they were challenged with HDM. Twenty-four dogs with a change in lesion score of 10 are included in this study.

The 24 dogs are randomly divided into 4 treatments groups (i.e. Placebo, Cortavance DDPA Treatment A and DDPA Treatment B), 3 incomplete crossover challenge phases, and 12 unique sequences (1-12) shown in Table 5 below. The first phase of the HDM challenges begins on Day 0, and the second and third phases begins on Days 35 and 70, respectively. The person(s) administering the test article are unblinded. Personnel evaluating skin lesions are blinded to treatment. The study design is composed of mutually orthogonal Latin squares with characteristics of a generalized Youden design that also balances for first-order carryover effects.

TABLE 5
Randomization of treatments

| | Challenge Period (treatment group) | | | Animal |
|---|---|---|---|---|
| Sequence | Phase I | Phase II | Phase III | ID |
| 1 | Placebo | Cortavance | Treatment A | 18 |
| 2 | Cortavance | Placebo | Treatment B | 6 |
| 3 | Treatment A | Treatment B | Placebo | 15 |
| 4 | Treatment B | Treatment A | Cortavance | 2 |
| 5 | Placebo | Treatment A | Treatment B | 17 |
| 6 | Cortavance | Treatment B | Treatment A | 14 |
| 7 | Treatment A | Placebo | Cortavance | 10 |
| 8 | Treatment B | Cortavance | Placebo | 7 |
| 9 | Placebo | Treatment B | Cortavance | 12 |
| 10 | Cortavance | Treatment A | Placebo | 22 |
| 11 | Treatment A | Cortavance | Treatment B | 16 |
| 12 | Treatment B | Placebo | Treatment A | 23 |

1. A composition comprising Dodeca-2,4,6,8,10-pentaenoic acid (DDPA) and an at least one excipient selected from the group consisting of diluents, fillers, disintegrants, binders, lubricants, surfactants, hydrophobic vehicles, water soluble vehicles, co-solvents, emulsifiers, buffers, humectants, moisturizers, solubilizers, preservatives, colorants, plasticizers, carriers, penetration enhancers, or combinations thereof.

2. The composition of claim 1, wherein said composition is an emulsion.

3. The composition of claim 2, wherein said emulsion of DDPA is a topical formulation for application to the skin of an animal, said topical formulation comprising a spray, an ointment, a cream, a foam, or a gel.

4. The composition of claim 3, wherein said topical formulation is for use in the preparation of a medication for the treatment of an inflammatory skin condition.

5. The composition of claim 1, wherein said DDPA comprises a therapeutically effective amount.

6. The composition of claim 5, wherein said DDPA comprises 0.01% to about 5% by weight of said composition.

7. The composition of claim 2, wherein said emulsion comprises an oil phase, a water phase, and a surfactant.

8. The composition of claim 2, wherein said emulsion is self-emulsifying.

9. The composition of claim 2, said composition is an oil-in-water emulsion.

10. The composition of claim 2, wherein said composition is a water-in-oil emulsion.

11. The composition of claim 1, wherein the pH of said composition is between 6 and 8.

12. The composition of claim 1, wherein said excipients are acceptable pharmaceutically, cosmetically, or both.

13. The composition of claim 1, wherein said excipient comprises an antioxidant, a preservative, a penetration enhancer, a chelating agent, pH adjusting agent, a humectant, a gelling agent, fragrance, a colorant, and combinations thereof.

14. The composition of claim 1, wherein said composition comprises a light protective excipient.

15. The composition of claim 1, wherein said composition is a homogenous emulsion

16. The composition of claim 2, wherein said emulsion comprises average oil droplet size of less than 35 microns.

17. The composition of claim 2, wherein said composition comprises said emulsion of DDPA and a hydrogel formed from a polymer, polyhydric alcohol, and water, wherein water is present in an amount of about 50% to about 99% by weight.

18. The composition of claim 2, wherein said composition comprises a cream formed from said emulsion of DDPA and comprises a hydrogel comprising a polymer, polyhydric alcohol, and water, wherein water and volatiles are present in an amount of less than 20% by weight and less than 50% hydrocarbons, waxes, or polyols as excipients.

19. The composition of claim 2, wherein said composition comprises an ointment formed from said emulsion of DDPA wherein said ointment comprises less than 20% water and volatiles, and greater than 50% hydrocarbons, waxes, or polyols, as excipients.

20. The composition of claim 1, further comprising an isolated Lactobacillus reuteri strain that does not produce DDPA.

21. The composition of claim 20, wherein said isolated Lactobacillus reuteri strain that does not produce DDPA is Lactobacillus reuteri strain 3630, a representative sample of cells of said strain having been deposited with the ATTC under ATCC Accession No PTA-126787.

22. A method of treating an inflammatory skin condition in animal in need thereof comprising applying a composition comprising an emulsion of Dodeca-2,4,6,8,10-pentaenoic acid (DDPA) and one or more excipients selected from the group consisting of diluents, fillers, disintegrants, binders, lubricants, surfactants, hydrophobic vehicles, water soluble vehicles, emulsifiers, buffers, humectants, moisturizers, solubilizers, preservatives, colorants, plasticizers, carriers, excipients, or combinations thereof.

23. The method of claim 22, wherein said condition comprises atopic dermatitis (AD), psoriasis (Ps), contact dermatitis, hidradenitis suppurativa (HS), pyoderma gangrenosum (PG), palmoplantar pustulosis (PPP), prurigo nodularis (PN), Sweet's syndrome, mutations in proline-serine-threonine phosphatase interacting protein 1 (PSTPIP1) (PAPA syndrome) PAPSH syndrome and PASH syndrome), Bechet's disease, bullous pemphigold, mucous membrane pemphigold, pemphis vulgaris, cutaneous Crohn's disease, Sjögren syndrome, systemic lupus erythematosus, pityriasis lichenoides chronica, and erythoderma.

24. The method of claim 22, wherein said composition is applied twice daily, once daily, once every other day; twice weekly; three times weekly, or once weekly.

25. The method of claim 22, wherein the production or expression of one or more proinflammatory cytokine in the skin of the treated animal is reduced.

26. The method of claim 22, wherein said production or expression of one or more proinflammatory cytokine selected from IL-6, TNF-α, IFN-γ and IL-1, including IL-1B, is reduced in the skin of a treated animal.

27. The method of claim 22, wherein said production or expression of one or more anti-inflammatory cytokine is increased in the skin of the treated animal.

28. The method of claim 22, wherein said production or expression of one or more anti-inflammatory cytokine selected from IL-10 and IL-22 is increased in the skin of the treated animal.

29. A method for treating an inflammatory skin disease comprising applying a topical composition to the skin of an animal subject in need of treatment, wherein said topical composition comprises an effective amount of an emulsion of Dodeca-2,4,6,8,10-pentaenoic acid (DDPA) and a pharmaceutically acceptable carrier.

30. The method of claim 29, wherein said inflammatory skin disease is selected from dermatitis, dermatitis, alopecia, and psoriasis, pyoderma gangrenosum, palmoplantar pustulosis, prurigo nodularis, or hidradenitis suppurativa.

31. The method of claim 29, wherein said composition further comprises administering one or more of an anti-inflammatory agent, molecule, or cytokine.

32. The method of claim 29, wherein the animal is a farm animal, a food production animal, domestic animal, or poultry

33. The method of claim 29, wherein the animal is a human.

34. The method of claim 29, wherein the animal is a horse, dog, or cat.

35. A method for modifying the expression of one or more proinflammatory cytokines or one or more anti-inflammatory cytokine in the skin of an animal in need thereof comprising administering to the animal a composition comprising effective amount of an emulsion of Dodeca-2,4,6,8,10-pentaenoic acid (DDPA) and a pharmaceutically acceptable carrier.

36. The method of claim 35, wherein said modifying is reducing the production or expression of one or more proinflammatory cytokines.

37. The method of claim 35, wherein said modifying is increasing the production or expression of one or more anti-inflammatory cytokine.

38. The method of claim 35, wherein the production or expression of one or more proinflammatory cytokine selected from IL-6, TNF-α, IFN-γ and IL-1, including IL-1B, is decreased in the skin of the animal.

39. The method of claim 35, wherein the production or expression of one or more anti-inflammatory cytokine selected from IL-10 and IL-22 is increased in the skin of the animal.

40. The method of claim 35, wherein said animal in need thereof has an inflammatory skin disease selected from dermatitis, dermatitis, alopecia, and psoriasis, pyoderma gangrenosum, palmoplantar pustulosis, prurigo nodularis, or hidradenitis suppurativa.

41. The method of claim 35, comprising further administering one or more of an anti-inflammatory agent, molecule or cytokine to the subject or animal.

42. The method of claim 35, wherein the composition comprising an effective amount of an emulsion of Dodeca-2,4,6,8,10-pentaenoic acid (DDPA) and a pharmaceutically acceptable carrier is administered prior to, in conjunction with, and/or following one or more anti-inflammatory agent, molecule, or cytokine, or immunomodulatory agent, molecule or cytokine

43. The method of claim 42, wherein the one or more anti-inflammatory agent, molecule or cytokine is selected from one or more anti-inflammatory, nonsteroidal anti-inflammatory drug (NSAID), steroid, biologic, antibiotic, or anti-diarrheal agent.

44. A composition for topical use in treating an inflammatory skin condition comprising an emulsion of Dodeca-2,4,6,8,10-pentaenoic acid (DDPA) and one or more excipients selected from the group consisting of diluents, fillers, disintegrants, binders, lubricants, surfactants, hydrophobic vehicles, water soluble vehicles, emulsifiers, buffers, humectants, moisturizers, solubilizers, preservatives, colorants, plasticizers, carriers, excipients, or combinations thereof.

45. The composition for topical use of claim 44, wherein said composition is an ointment, a cream, a foam, a gel, or a spray

46. The composition for topical use of claim 44, wherein said composition is an oil-in-water emulsion.

47. The composition for topical use of claim 44, wherein said composition is a water-in-oil emulsion.

48. The composition for topical use of claim 44, wherein said ointment, cream, foam, or gel is hydrophobic or lipophilic.

49. The composition for topical use of claim 44, wherein said ointment, cream, foam, or gel is applied to a patch.

50. The composition for topical use of claim 44, wherein said inflammatory skin disease is selected from dermatitis, dermatitis, alopecia, and psoriasis, pyoderma gangrenosum, palmoplantar pustulosis, prurigo nodularis, or hidradenitis suppurativa.

51. The composition for topical use of claim 44, wherein said inflammatory disease is selected from the group consisting of atopic dermatitis, seborrheic dermatitis, and psoriasis.

52. The composition for topical use of claim 44, wherein said composition comprises a light protective component.

53. The composition for topical use of claim 52, wherein said light protective component comprises zinc oxide or titanium oxide.

54. The composition for topical use of claim 44, wherein said composition is opaque.

55. The composition for use of claim 44, further comprising an isolated Lactobacillus reuteri strain that does not produce DDPA.

56. The composition for use of claim 55, wherein said isolated Lactobacillus reuteri strain that does not produce DDPA is Lactobacillus reuteri strain 3630, which corresponds to ATCC Patent Deposit Number PTA-126787.

57. The composition for use of claim 44, wherein the production or expression of one or more proinflammatory cytokine in the skin of the animal is reduced.

58. The composition for use of claim 57, wherein the production or expression of one or more proinflammatory cytokine that is reduced selected from IL-6, TNF-α, IFN-γ and IL-1, including IL-1B, is reduced in the skin system of the animal.

59. The composition for use of claim 44, wherein the production or expression of one or more anti-inflammatory cytokine is increased in the skin of the animal.

60. The composition for use of claim 59, wherein the production or expression of one or more anti-inflammatory cytokine selected that is increased is IL-10, IL-22, or both, is increased in the skin of the animal.

61. The composition for use of claim 44, wherein the animal is bird, poultry, a human, or a non-human mammal.

62. The composition for use of claim 61, wherein the animal is a farm animal or food production animal, a domestic animal, or wherein the bird is poultry

63. The composition for use of claim 61, wherein the animal is a human.

64. The composition for use of claim 61, wherein the animal is a horse, dog, or cat.
